(19) United States
(12) Patent Application Publication
(10) Pub. No.: US 2025/0290917 A1
(43) Pub. Date: Sep. 18, 2025

(54) MAPPING METABOLITE- AND METAL ION-PROTEIN INTERACTOMES USING FUNCTIONAL DNA-BASED PROXIMITY LABELING

(71) Applicant: Board of Regents, The University of Texas System, Austin, TX (US)

(72) Inventors: Yi LU, Austin, TX (US); Mandira BANIK, Austin, TX (US)

(73) Assignee: Board of Regents, The University of Texas System, Austin, TX (US)

(21) Appl. No.: 19/080,132

(22) Filed: Mar. 14, 2025

Related U.S. Application Data
(60) Provisional application No. 63/566,229, filed on Mar. 16, 2024.

(51) Int. Cl. G01N 33/53; G01N 33/542; G01N 33/68; G01N 33/84

(52) U.S. Cl. CPC G01N 33/5308; G01N 33/542; G01N 33/6848; G01N 33/84

(57) ABSTRACT

Provided herein are methods of detecting macromolecule (e.g., proteins or nucleic acids) interactors in the vicinity of a metabolite or metal ion pool in a cell. The methods may comprise DNAzyme-and aptamer-based proximity labeling identification (DAP-ID) to identify macromolecules (e.g., proteins or nucleic acids) in the vicinity of intracellular metabolites or metal ions. DAP-ID leverages metabolite-selective aptamers and metal ion-selective DNAzymes that undergo target-induced conformational changes to expose reactive electrophiles (e.g., sulfonyl fluoride electrophiles) for covalent macromolecule (e.g., protein or nucleic acid) tagging.

### REFERENCE TO RELATED APPLICATIONS

The present application claims the priority benefit of U.S. provisional application No. 63/566,229, filed Mar. 16, 2024, the entire contents of which are incorporated herein by reference.

### STATEMENT OF FEDERALLY SPONSORED RESEARCH

This invention was made with government support under Grant no. R35 GM141931 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on Mar. 12, 2025, is named UTSBP1368US.xml and is 30,721 bytes in size.

### BACKGROUND

### 1. Field

The present disclosure relates generally to the field of cellular biology. More particularly, it concerns metabolite-and metal ion-protein interactomes.

### 2. Description of Related Art

Cellular functions are regulated by complex interplays between proteins and metabolites and between proteins and metal ions. These metabolites and metal ions influence protein function through a range of covalent and noncovalent interactions. For example, ATP hydrolysis can result in protein phosphorylation to drive signal transduction (Jelcic et al., 2020), while acetyl COA is required for histone acetylation to regulate gene expression (Trefely et al., 2022). Similarly, in addition to serving as cofactors in all metalloenzymes, metal ions, such as sodium, allosterically regulate ion channels (Agasid et al., 2021), while calcium activates extensive protein-protein interactions (Chin et al., 2000). Moreover, intracellular imaging techniques have found that metabolites and metal ions, such as ATP and Na⁺, are often distributed inhomogeneously within the cell, likely due to different interactions with their physiological partners, such as nearby proteins, to enable biological functions (Dean et al., 2012; Imamura et al., 2009; Meyer et al., 2019). Despite their importance, many of these interactions remain undiscovered and underexplored within complex cellular microenvironments, due to a lack of methods for generalizable and discovery-based approaches to identify these interactions in live cells. This is in direct contrast to the numerous platforms for the identification of protein-protein (Cho et al., 2020; Rhee et al., 2013; Geri et al., 2020; Roux et al., 2012) and protein-DNA/RNA interactions (Padron et al., 2019; Wei et al., 2023).

Because metabolites and metal ions are low molecular weight and chemically similar to off-target species, and their interactions with proteins can be transient and low-affinity, tools to detect protein-protein interactions can be difficult to generalize to protein-metabolite and protein-metal ion interactions (Kim et al., 2024). To address this issue, equilibrium dialysis, and chemical modulation methods have been developed (Hicks et al., 2023; Piazza et al., 2018; Zeng et al., 2024). However, these methods rely on cell lysates, which remove the spatial interactions from their native cellular contexts, losing the context of varied metabolite and metal ion intracellular distributions. In addition, mass spectrometry imaging methods provide high throughput imaging of metabolites and proteins, but they use fixed cells, and their resolution cannot determine subcellular microenvironments. In live cells, chemoproteomics strategies using derivatized metabolite analogs have been applied for metabolite-protein discovery, but these are constrained by knowledge of well-defined metabolite binding pockets, predominantly performed with cell lysates, and often limited to labeling low abundant surface exposed residues (Qin et al., 2020). The interactions of metabolites and metal ions with their protein partners are dynamic (Lindsley et al., 2006), lack consensus motifs for binding regions (Adachi et al., 2014), and occur through a wide range of covalent and non-covalent interactions. Ultimately, these challenges have limited the development of discovery-based and generalizable methods to identify proteins in the vicinity of metabolite and metal ion microenvironments.

Among the platforms for identifying protein-protein and protein-DNA/RNA interactions, proximity labeling has been a powerful method (Milione et al., 2024; Hanswillemenke et al., 2024; Pani et al., 2024; Myers et al., 2018). However, methods for applying proximity labeling to discovering protein-metabolite and protein-metal ion interactions are needed.

### SUMMARY

Provided herein are methods of detecting macromolecule (e.g., proteins or nucleic acids) interactors in the vicinity of a metabolite or metal ion pool in a cell, the methods comprising:
(a) administering to the cell a conformationally gated sensor that has affinity for the metabolite or metal ion, wherein the conformationally gated sensor comprises a hidden reactive (e.g., protein-reactive) electrophile;
(b) incubating the cell under conditions to allow the sensor to interact with the metabolite or metal ion, thereby undergoing a conformational change upon binding the metabolite or metal ion, thereby exposing the reactive (e.g., protein-reactive) electrophile, wherein the exposed reactive (e.g., protein-reactive) electrophile can label nearby macromolecules (e.g., proteins or nucleic acids); and
(c) extracting and identifying the proximal macromolecules (e.g., proteins or nucleic acids) that have been labeled by the sensor's reactive (e.g., protein-reactive) electrophile.

The conformationally gated sensor may be an aptamer. The aptamer may comprise a fluorescent tag. The fluorescent tag may be fluorescein.

The methods provided herein may further comprise a photocaged strand complementary to the aptamer, and wherein the photocaged complementary strand may comprise a photocleavable o-nitrobenzyl group, and wherein the aptamer may hybridize with the photocaged strand generating a protected aptamer to prevent the aptamer from premature response to the metabolite or metal ions, and to protect the reactive (e.g., protein-reactive) electrophile from covalent labeling of reactive nucleophiles. The photocaged complementary strand may comprise a quencher.

The methods may further comprise exposing the protected aptamer to a light stimulus, wherein the exposing to the light stimulus may decage the aptamer, thereby allowing binding of the aptamer to the metabolite or metal ion.

The conformationally gated sensor may be a DNAzyme. The DNAzyme may comprise an enzyme strand and a substrate strand. The substrate strand may comprise a photocleavable o-nitrobenzyl group.

The method may further comprise exposing the DNAzyme to a light stimulus, wherein the exposing to the light stimulus may decage the substrate strand, thereby allowing cleavage of the substrate strand upon binding of the metabolite or metal ion.

The substrate strand may comprise at least one ribonucleotide.

The binding of the metabolite or metal ion to the DNAzyme may lead to cleavage of the substrate strand by the enzyme strand, whereby the cleavage may lead to exposure of the reactive (e.g., protein-reactive) electrophile.

The enzyme strand may comprise a fluorescent tag. The fluorescent tag may be fluorescein. The substrate strand may comprise a quencher.

The hidden reactive electrophile may be an electrophilic sulfonyl fluoride group. The electrophilic sulfonyl fluoride group may be at the 2′ sugar position of a DNA base in the sensor.

The conformationally gated sensor may comprise a biotin. Extracting the labeled macromolecules (e.g., proteins or nucleic acids) may comprise pulling down the labeled macromolecules (e.g., proteins or nucleic acids) using streptavidin.

Identifying the proximal macromolecules (e.g., proteins or nucleic acids) may comprise quantitative mass spectrometry.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF DRAWINGS

The patent or application file contains at least one drawing executed in color. Copies of this patent or patent application publication with color drawing(s) will be provided by the Office upon request and payment of the necessary fec.

The following drawings form part of the present specification and are included to further demonstrate certain aspects of the present invention. The invention may be better understood by reference to one or more of these drawings in combination with the detailed description of specific embodiments presented herein.

FIG. 1 depicts Proximity-based protein tagging upon a metabolite-specific or metal ion-specific sensor structural switch. Metabolites and metal ions affect protein structure and activity through multiple mechanisms. The cellular environment contains a rich mixture of macromolecules, free and loosely bound metabolites and metal ions, and their interactions with one another. Proximity labeling in live intracellular environments is performed by using sensors composed of nucleic acids, such as aptamers (targets metabolites) and DNAzymes (targets metal ions), with rationally designed electrophilic modifications. These sensors undergo a conformational change upon target binding. Prior to target binding, the aptamer/DNAzyme sensor hides the electrophile within its duplex. Upon probe activation and sensor structural switch, the strands dehybridize, resulting in exposure of the electrophile and subsequent protein tagging.

FIGS. 2A-F depict the design and demonstration of DAP-ID using a modified ATP aptamer. (A) The design of the ATP aptamer-guided DAP-ID consists of an ATP aptamer (purple) (SEQ ID NO: 7), modified with aryl SF groups for protein tagging, a fluorescein at the 5′ end of the aptamer strand paired with a Black Hole Quencher-1 at the 3′ end of the complementary strand for sensor visualization, and a 3′ biotin in the aptamer strand as a handle to tag cross-linked proteins. The aptamer strand is hybridized with a photocaged complementary strand (SEQ ID NO: 10 and SEQ ID NO: 11) to both protect the aptamer from premature response to ATP and to protect the SF electrophile from covalent labeling of reactive nucleophiles. Upon a light stimulus, the decaged hybridized sensor is available for response to ATP. Upon exposure to ATP, the sensor undergoes a structural switch, which exposes the SF electrophile for protein tagging and results in a fluorescence signal change. (B) The modified ATP aptamer maintains its metabolite binding properties in response to a physiologically relevant range of ATP, while the scrambled negative control sequence displays no fluorescence change. (C) Electrophoretic mobility shift assay corresponding to model protein labeling in vitro only with the addition of the 2′-SF-modified active ATP aptamer and ATP. NH2 modifications in the place of the SF account for non-covalent crosslinking background, while inactive controls account for background labeling. 2 biological replicates were performed. (D) Confocal imaging of the ATP aptamer sensor (green) and ATP aptamer-guided labeled proteins (cyan) in HepG2 cells compared to the inactive and non-covalent amine controls. Scale bars are 20 μm. Statistical significance is a 2-tailed student's t-test with 5 biological replicates. Error bars are the standard deviation of the mean. (E) Western blot with crude cell lysate after delivering the controls and ATP aptamer-guided DAP-ID platform to evaluate the degree of biotinylation. 2 biological replicates were performed. (F) Quantitative proteomics with the ATP aptamer DAP-ID platform significantly enriches known ATP-related proteins, with 97% of the proteins identified already established to be directly related to ATP (gold) or interactors of ATP-related proteins (blue). The volcano plot shows averaged log₂ ratio on the x-axis and the false discovery rate corrected P-value on the −log₁₀ scale. Proteomics was performed with 3 biological replicates.

FIG. 3 depicts an exemplary synthesis reaction for NHS-SF.

FIG. 4 depicts NMR for product verification of NHS-SF.

FIG. 5 depicts MALDI-TOF-MS of active ATP aptamer pre-SF modification (mass 11005.01) and post-SF modification (11327.380).

FIG. 6 depicts MALDI-TOF-MS of active Na+-DNAzyme pre-SF modification (mass 189783.6) and post-SF modification (19276.975).

FIG. 7 depicts modeling of SF-incorporated strand hybridization. The p-arylsulfonyl fluoride was manually docked at the 2′ ribose position of the model 8-17 DNAzyme (PDB 5XM8) in PyMol. SF's aromatic ring shown in cyan with distances to adjacent bases drawn in yellow.

FIG. 8 depicts the predicted secondary structure. ATP aptamer folding with modified bases shown in black boxes (SEQ ID NO: 7).

FIG. 9 depicts strand hybridization using native PAGE. ATP aptamer hybridization with its complementary quencher strand reaches completion, so no free ATP aptamer with the SF modification is exposed.

FIG. 10 depicts representative raw images for ATP aptamer quantification. The NH2-modified photocaged scrambled control, NH2-modified photocaged active ATP aptamer, the SF-modified photocaged scramble control, and the SF-modified photocaged active ATP aptamer into HepG2 cells using Turbofect. One control group of no uncaging was used to evaluate sensor photoactivation (left). After 4 hours, cells were washed to remove undelivered strands and strands were decaged with light (right). After 4 hours, cells were washed to remove undelivered strands and strands were decaged with light. After an additional 4 hours, cells were fixed, permeabilized, and stained with Cy5-streptavidin. Confocal imaging of all sample groups visualized fluorescein (green) on the DNA strands, streptavidin (magenta) for labeled proteins, and brightfield for cell morphology and sensor internalization. Scale bar is 20 μmeter.

FIG. 11 depicts MTT cell toxicity assay. To compare the effects of the delivered DNA and photoactivation on cell viability, HepG2 cells were incubated with either no variables (no DNA), the SF-modified ATP aptamer sensor delivered through Turbofect, or hydrogen peroxide for 4 hours in two groups. After replacing the media, one group was then irradiated with 365 nm for 20 minutes. MTT assays found DNA, with and without light photoactivation, had minimal effects on cell viability. The positive control of hydrogen peroxide resulted in almost complete cell death

FIG. 12 depicts full western blots for ATP aptamer-PL system. Top image corresponds to streptavidin-IR800 stained blot, while the bottom image corresponds to the total protein stain loading control.

FIG. 13 depicts streptavidin bead pulldown elution. Following the pulldown with streptavidin magnetic beads procedure, the entire elution for each sample was run on a 4-20% SDS-PAGE gel and transferred for Western blot analysis with streptavidin staining.

FIG. 14 depicts FRAP fluorescence recovery (top) and video snapshots (bottom) to measure the DNA diffusion coefficient. Three regions of interest were applied with equal size circular regions for all three spots. A sample of interest (red), a similar fluorescent intensity reference sample (blue), and a background in a location with no cells (green) were used with a radius of 680 nm. As shown from these snapshots, fluorescence intensity in the sample of interest spot failed to achieve fluorescence recovery, while the control similar reference spot faced negligible photobleaching during the imaging period. Scale bar is 10 μmeter.

FIGS. 15A-D depicts the design and demonstration of DAP-ID using Na+-DNAzyme. (A) The design of the Na+-DNAzyme-guided DAP-ID platform consists of a Na+-DNAzyme (SEQ ID NO: 1), modified with aryl SF groups, a fluorophore and quencher for sensor visualization, and a biotin for tagged protein identification. The enzyme strand is hybridized with a photoprotecting nitrobenzyl-modified RNA-containing substrate strand (SEQ ID NO: 5). After light activation, the photoprotecting group is removed. In the presence of Na+, the sensor undergoes a cleavage reaction of the substrate strand into two strands (SEQ ID NO: 16 and SEQ ID NO: 17), exposing the SF electrophile, and resulting in fluorescence signal change. The SF is then accessible to nearby proteins for covalent bond formation. Tagged proteins are purified with the biotin tag. (B) Confocal imaging of the Na+-DNAzyme (green) and Na+-DNAzyme-guided labeled proteins (cyan) in HepG2 cells compared to the inactive and non-covalent NH2 controls. Imaging scale bars are 20 μm. Statistical significance is a 2-tailed student's t-test with 5 biological replicates. Error bars are standard deviation of the mean. (C) Western blot with crude cell lysate after delivering the controls and Na+-DNAzyme-guided DAP-ID platform to evaluate degree of biotinylation. Major bands are native biotinylated proteins. 2 biological replicates were performed. (D) Quantitative proteomics with the Na+-DNAzyme-PL platform significantly enrich known Na+-related proteins, with 70% of the proteins identified already established to be directly related to Na+or interactors of Na+-related proteins. The volcano plot shows averaged log2 ratio on the x-axis and the false discovery rate corrected P-value on the −log10 scale. Proteomics was performed with 3 biological replicates.

FIG. 16 depicts predicted secondary structure. Na+-DNAzyme enzyme strand folding with modified bases shown in black boxes (SEQ ID NO: 1).

FIG. 17 depicts fluorescence spectroscopy of the Na+-DNAzyme response. The 2′-SF modified active Na+-DNAzyme cleaves its complementary substrate strand in the presence of sodium, while the 2′-SF modified point mutation control does not exhibit a sodium response.

FIG. 18 depicts representative raw images for Na+-DNAzyme quantification. The amine modified photocaged point mutation control, amine modified photocaged active Na+-DNAzyme, the SF-modified photocaged point mutation control, and the SF-modified photocaged active Na+-DNAzyme were delivered into HepG2 cells using Turbofect. One control group of no uncaging was used to evaluate sensor photoactivation (left). After 4 hours, cells were washed to remove undelivered strands and strands were decaged with light (right). After an additional 4 hours, cells were fixed, permeabilized, and stained with Cy5-streptavidin. Confocal imaging of all sample groups visualized fluorescein (green) on the DNA strands, and streptavidin (magenta) for labeled proteins, and brightfield for cell morphology and sensor internalization. Scale bar is 20 μmeter.

FIG. 19 depicts strand hybridization using native PAGE. Na+-DNAzyme enzyme strand hybridization with its complementary substrate strand reaches completion, so no free Na+-DNAzyme with the SF modification is exposed.

FIG. 20 depicts uncropped western blots for Na+-DNAzyme-PL system. Top image corresponds to streptavidin-IR800 stained blot, while the bottom image corresponds to the total protein stain loading control.

FIGS. 21A-D depict the validation of a previously unknown sodium-protein relationship. (A) Colocalization of the Na+-DNAzyme (green), Na+-DNAzyme-guided labeled proteins (cyan), ACAA2 immunostaining (red), and the composite. 3 technical replicates were performed. (B) Other top protein candidates' composite images and corresponding Pearson's correlation values. 3 technical replicates were performed. (C) Kinetics of ACAA2 activity in the presence of sodium versus potassium. 3 biological replicates were performed (D) Measuring global intracellular sodium pool changes after siRNA-mediated knockdown of ACAA2 in HepG2 cells. 5 biological replicates were performed. Imaging scale bars are 20 μm. Statistical significance was determined from a 2-tailed student's t-test. Error bars are standard deviation of the mean.

### DETAILED DESCRIPTION

A proximity labeling platform to discover macromolecule (e.g., proteins or nucleic acids)-metabolite/metal ion microenvironments needs to first recognize a specific metabolite or metal ion and subsequently tag surrounding macromolecules (e.g., proteins or nucleic acids). For the metabolite or metal ion recognition, functional nucleic acids, such as metabolite-selective aptamers (Huizenga et al., 1995; Yu et al., 2021; Nakatsuka et al., 2018; Xie et al., 2020) and metal ion-selective DNAzymes (Breaker et al., 1994; McGhee et al., 2021; Torabi et al., 2015; Li et al., 2023; Zhou et al., 2017), offer unique advantages, as they are generated through in vitro selection to bind almost any metabolite or metal ion with high selectivity, and undergo a structural switch upon binding their respective target (Munzar et al., 2018). Aptamers are usually hybridized with a partially complementary strand that is released upon target metabolite binding. Similarly, DNAzymes are normally hybridized with a complementary substrate strand, containing a single ribonucleotide. In the presence of the target metal ion, the substrate strand may be cleaved at the ribonucleotide, decreasing the substrate strand's melting temperature, resulting in strand dehybridization (Breaker et al., 1994). In both cases, after the target-induced conformational change, the double-stranded sensor may unwind into single strands.

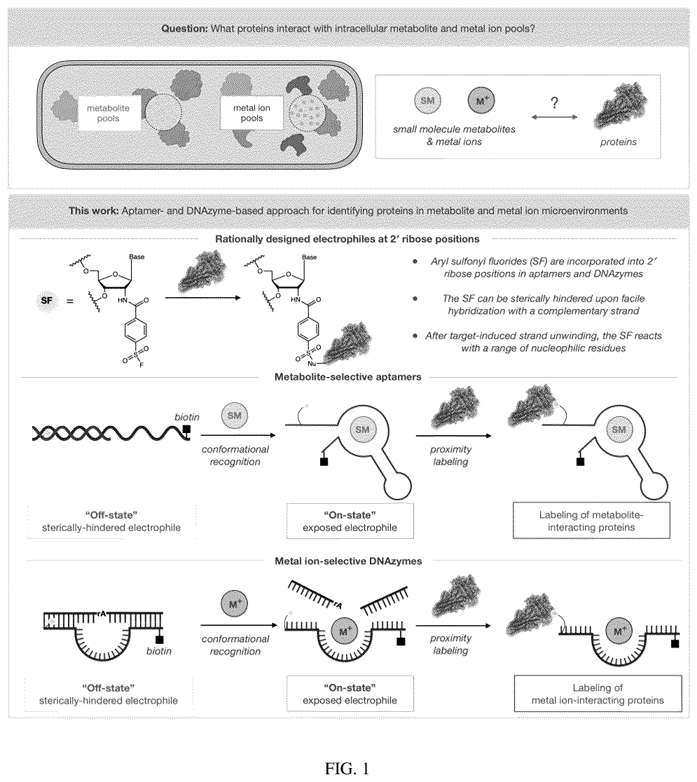

For macromolecule (e.g., protein or nucleic acid) tagging, the 3D reconfiguration of aptamers and DNAzymes was utilized to embed an electrophilic sulfonyl fluoride (SF) group at 2′ ribose positions of certain nucleotides. In the absence of the target, the electrophile is protected by the sensor's duplex structure. After the target-induced conformational change, the SF can be selectively unveiled for protein tagging (FIG. 1). The SF electrophile is ideal for this purpose because it is a low molecular weight small molecule, allowing for its masked reactivity in the sensor's hybridized off-state. Moreover, the SF reaction occurs readily under biological conditions, may require a hydrogen bonding network for covalent bond formation, and reacts with many nucleophilic amino acids (Barrow et al., 2019; Qin et al., 2023). These properties increase the SF's likelihood to label macromolecules (e.g., proteins or nucleic acids) proximal to the site of sensor activation and to decrease labeling bias compared to residue-specific strategies, which may miss proteins with low expression of reactive surface residues (Gould et al., 2015).

Provided herein is DNAzyme-and aptamer-based proximity labeling identification (DAP-ID) to identify macromolecule (e.g., proteins or nucleic acids) interactors in the vicinity of intracellular endogenous metabolites and metal ions. DAP-ID was developed as a generalizable method which may be used to identify macromolecule (e.g., proteins or nucleic acids)-metabolite or macromolecule (e.g., proteins or nucleic acids)-metal ion interactomes in the spatial microenvironment of live cells. DAP-ID may embed electrophilic SF groups in a metabolite-selective aptamer or a metal ion-selective DNAzyme. Upon target binding, both sensor classes may dehybridize and unveil the SF groups, resulting in spatial labeling of unique protein pools adjacent to physiologically important metabolites and metal ions.

### I. Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein “nucleic acid” generally refers to a polymeric form of nucleotides of any length (e.g. at least 2, 3, 4, 5, 6, 10, 50, 100, 200, 500 or 1000 nucleotides), either deoxyribonucleotides or ribonucleotides or a combination thereof, and any modifications thereof. Modifications include, but are not limited to, those that provide other chemical groups that incorporate additional charge, polarizability, hydrogen bonding, electrostatic interaction, and fluxionality to the nucleic acid ligand bases or to the nucleic acid ligand as a whole. Accordingly, the nucleic acids described herein include not only the standard bases adenine (A), cytosine (C), guanine (G), thymine (T), and uracil (U) but also non-standard or non-natural nucleotides, analogs and derivatives thereof. Non-standard or non-natural nucleotides such as isoC or isoG, are described, for example, in U.S. Pat. Nos. 5,432,272, 5,965,364, 6,001,983, 6,037,120, and 6,140,496, all of which are incorporated herein by reference and include bases other than A, G, C, T, or U that can be incorporated into a growing nucleic acid strand by a polymerase and are capable of base-pairing with a complementary non-standard or non-natural nucleotide to form a base pair.

An “aptamer” refers to a polynucleotide which contains an effector binding site. An “effector binding site” may be “specific,” that is, binding only one effector molecule in the presence of other effector molecules. An example of effector binding site specificity is when only an adenosine molecule binds in the presence of many other similar molecules, such as cytidine, gaunosine and uridine. Alternatively, an effector binding site may be “partially” specific (binding only a class of molecules), or “non-specific” (having molecular promiscuity).

### II. Nucleic Acid Enzymes

A variety of nucleic acid enzymes have been discovered or developed that can be used with the methods provided herein, including those described in US2009/0011402,US2006/0094026, and U.S. Pat. No. 20,040,175693, which are incorporated herein by reference. The catalytic activity of the nucleic acid enzymes may depend on or require one or more co-factors, such as a metal ion. In vitro selection may be used to “enhance” selectivity and sensitivity for a particular ion. In some preferred embodiments, the nucleic acid enzymes catalyze a molecular dissociation (cleavage or transfer). In some preferred embodiments, the nucleic acid enzyme results in self-cleavage of the nucleic acid enzyme (e.g., DNAzyme) such that the reaction products can be detected to quantify the presence of the co-factor (e.g., metal ion) that may be present in a biological sample (e.g., tissue sample). The nucleic acid enzyme may preferably contain an affinity tag (e.g., a poly-A tail, or biotin) that may allow for improved purification and sequencing of the reaction products generated by exposure to the co-factor (e.g., metal ion). In some preferred embodiments, the nucleic acid enzyme (e.g., DNAzyme) is photocaged, and such metal-dependent DNAzymes may be used for the quantitative detection of metal ions, e.g., in living cells (Hwang et al., 2019).

A nucleic acid enzyme that catalyzes the cleavage of a nucleic acid in the presence of an effector is preferably used in methods provided herein. The nucleic acid enzyme may be DNA (deoxyribozyme). The DNAzyme may be modified or include an extended chemical functionality, e.g., as described in Santoro et al. (2000).

Methods of producing deoxyribozymes include chemical oligonucleotide synthesis, polymerase chain reaction (PCR), DNA cloning and replication. Preferably the nucleic acid enzymes are DNA. Nucleotides containing modified bases, phosphates, or sugars may also be used; in some instances, these modified nucleotides may be advantageous for stability or confer effector specificity. Examples of modified bases include inosine, nebularine, 2-aminopurine riboside, N⁷-deazaadenosine, and O⁶-methylguanosine (Earnshaw and Gait 1998). Modified sugars and phosphates include 2′-deoxynucleoside, abasic, propyl, phosphorothioate, and 2′-O-allyl nucleoside (Earnshaw and Gait 1998). DNAzymes can be used to detect a variety of cofactors for different purposes. DNAzymes that bind metal ions that influence transcriptomic activity, including heavy metal ions (e.g., lead, mercury, cadmium, chromium) as well as ions of iron, copper, zinc, can be used in the methods provided herein.

### III. Fluorescent Tags and Quenchers

As used herein, a “fluorescent dye” or “fluorophore”, or “fluorescent tag” is a chemical group that can be excited by light to emit fluorescence at a given wavelength or range of wavelengths. Dyes that may be used in the disclosed methods include, but are not limited to, fluorophores such as ALEXA FLUOR™ dyes, Fluorescein, HEX™ or AQUAPHLUOR® and others known to those skilled in the art. Examples of fluorophores include, a red fluorescent squarine dye such as 2,4-Bis[1,3,3-trimethyl-2-indolinylidenemethyl] cyclobutenediylium-1,3-dioxolate, an infrared dye such as 2,4 Bis[3,3-dimethyl-2-(1H-benz[e]indolinylidenemethyl)] cyclobutenediylium-1,3-dioxolate, or an orange fluorescent squarine dye such as 2,4-Bis[3,5-dimethyl-2-pyrrolyl] cyclobutenediylium-1,3-diololate. Additional non-limiting examples of fluorophores include quantum dots, ALEXA FLUOR® dyes (sulfonated amino-coumarin or rhodamine), aminomethylcoumarin (AMCA), BODIPY® (borondipyrromethene dye) fluorophores, including BODIPY® 630/650, BODIPY® 650/665, BODIPY®-FL, BODIPY®-R6G, BODIPY®-TMR, and BODIPY®-TRX, CASCADE BLUE® (pyrenyloxytrisulfonic acid), CYDYE™ (cyanine-based fluorophores), including but not limited to CY2™ (cyanine dye 2), CY3™ (cyanine dye 3), and CY5™ (cyanine dye 5), a DNA intercalating dye, 6-FAM™ (6-carboxyfluorescein), Fluorescein, HEX™ (hexachloro-fluorescein), 6-JOE™ (6-carboxy-4′,5′-dichloro-2′,7′-dimethoxyfluorescein), OREGON GREEN® fluorophores (fluorinated analogs of fluorescein), including OREGON GREEN® 488, OREGON GREEN® 500, and OREGON GREEN® 514, PACIFIC BLUE™ (3-carboxy-6,8-difluoro-7-hydroxycoumarin), REG™, phycobilliproteins including, but not limited to, phycoerythrin and allophycocyanin, RHODAMINE GREEN™ (CAS: 189200-71-3), RHODAMINE RED™ (a triarylmethane dye), ROX™ (carboxyrhoadmine), TAMRA™ (carboxytetramethylrhodamine), TET™ (tetrachlorofluorescein), Tetramethylrhodamine, or TEXAS RED® (sulforhodamine 101 acid chloride). A signal amplification reagent, such as tyramide (PerkinElmer), may be used to enhance the fluorescence signal.

It is also contemplated that fluorophore/quencher-based systems may be used with the methods and compositions disclosed herein. When a quencher and fluorophore are in proximity to each other, the quencher quenches the signal produced by the fluorophore. When the fluorophore/quencher pair are separated, the fluorophore may be able to emit a fluorescent signal. Fluorophore/quencher-based systems reduce background. The oligonucleotides and nucleotides of the disclosed methods may be labeled with a quencher, suitable for quenching fluorescence of a fluorophore. Quenching may include dynamic quenching (e.g., by FRET), static quenching, or both.

### IV. Photolabile Protecting Groups

A photolabile protecting group (PPG; also known as: photoremovable, photosensitive, or photocleavable protecting group) is a chemical modification to a molecule that can be removed with light. PPGs enable high degrees of chemoselectivity as they allow researchers to control spatial, temporal and concentration variables with light.

Nitrobenzyl-based PPGs are often considered the most commonly used PPGs. An incident photon (200 nm<λ<320 nm) breaks the N═O π-bond in the nitro-group, bringing the protected substrate into a diradical excited state. Subsequently, the nitrogen radical abstracts a proton from the benzylic carbon, forming the aci-nitro compound. Depending on pH, solvent and the extent of substitution, the aci-nitro intermediate decays at a rate of roughly 102-104 s⁻¹. Following resonance of the π-electrons, a five-membered ring is formed before the PPG is cleaved yielding 2-nitrosobenzaldehyde and a carboxylic acid.

Overall, nitrobenzyl-based PPGs are highly general. The list of functional groups that can be protected include, but are not limited to, phosphates, carboxylates, carbonates, carbamates, thiolates, phenolates and alkoxides. Additionally, while the rate varies with a number of variables, including choice of solvent and pH, the photodeprotection has been exhibited in both solution and in the solid-state. Under optimal conditions, the photorelease can proceed with >95% yield.

### V. Protein Reactive Electrophiles

A protein reactive electrophile is a chemical species that readily reacts with nucleophilic amino acid residues on a protein, forming a covalent bond. Sulfonyl fluorides are one example of an electrophile that can react with a broad range of amino acids, including cysteine, lysine, tyrosine, and histidine. These groups may react with and label proteins in close vicinity.

### VI. Metabolites and Metal-Ions

Metal ions are common cofactors. Metal ion cofactors may include iron, magnesium, manganese, cobalt, copper, zinc, calcium, sodium, or molybdenum. These metal ions interact with proteins in cells, which may affect protein activity and/or function.

Metabolites are also common cofactors in cells. A metabolite is an intermediate or product of metabolism. Metabolites may include, for example, small molecules. Metabolites have various functions, including fuel, structure, signaling, stimulatory and inhibitory effects on enzymes, catalytic activity of their own (usually as a cofactor to an enzyme), defense, and interactions with other organisms (e.g. pigments, odorants, and pheromones).

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

### A. Chemicals and General Instrumentation

All DNA strands were synthesized in house following the general DNA synthesis procedure. All standard monomers were purchased from Glen Research (Sterling, VA). For modifications, iSpPC was purchased from Glen Research, 2′ amino cytidine was purchased from ChemGenes (Wilmington, MA), and the photocaged rA base was made in house. MilliQ water was used for buffer preparation. Buffer reagents and chemicals were purchased from Sigma-Aldrich at the highest purity available. Antibodies were purchased from Thermo-Fisher and all other Western blot supplies were purchased from Bio-Rad. Western blots were run with 4-20% Bio-Rad precast gels and semidry transfer equipment. Fluorimetry experiments were performed with an ISS ChronosDFD fluorimeter (Urbana, IL). Confocal microscopy images were collected on Zeiss 710 laser scanning microscope (UT Austin Center for Biomedical Research Support). LC-MS. Small molecule mass spectrometry was performed through high-resolution Electrospray Ionization (Agilent Technologies 6546 Accurate-Mass Q-TOF LC/MS) by the Mass Spectrometry Facility of the Department of Chemistry at UT Austin. MALDI-TOF (Bruker Autoflex) operated in the linear TOF mode performed in the negative ion mode with a 1:1 solution of 0.7 M 3-hydroxypicolinic acid and 0.07 M ammonium citrate as the matrix. Quantitative proteomics (Thermo Fusion Orbitrap Lumos Tribrid) was performed by the UT Austin Biological Mass Spectrometry Facility (RRID: SCR_021728). NMR data collected with Agilent MR 400 NMR spectrometer in deuterated solvents from Cambridge Isotope Laboratories (Cambridge, MA), with chemical shifts reported in ppm.

Exemplary DNA sequences used are shown in Table 1.

TABLE 1
Sequences of DNA used. FAM is fluorescein, NH2 is amine
control, SF is the aryl sulfonyl fluoride attached through
the NH2, rA is the in house synthesized photocaged adenosine,
iSpPC is the photocleavable linker ([4-(4,4′-dimethoxytrityloxy)
butyramidomethyl)-1-(2-nitrophenyl)-ethyl]-2-cyanoethyl-
(N,N-diisopropyl)-phosphoramidite), BHQ1 is Black Hole
Quencher 1, and r indicates a ribonucleotide.

| Sequence Name | Sequence (5′ to 3′) |
|---|---|
| Sodium Active Enzyme | GGC GGT_{biotin} ACC AGG TCA AAG GTG GGT GAG GGG ACG |
| Amine (SEQ ID NO: 1) | CCA AGA GTC CCC GCG GTT AGA TC_{NH2}C_{NH2} A/BHQ1/ |
| Sodium Point Mutation | GGC GGT_{biotin} ACC AGG ACA AAG GTG GGT GAG GGG ACG |
| Enzyme (SEQ ID NO: 2) | CCA AGA GTC CCC GCG GTT AGA TC_{NH2}C_{NH2} A/BHQ1/ |
| Sodium Active Enzyme | GGC GGT_{biotin} ACC AGG TCA AAG GTG GGT GAG GGG ACG |
| SF (SEQ ID NO: 3) | CCA AGA GTC CCC GCG GTT ACA TC_{SF}C_{SF} A/BHQ1/ |
| Sodium Point Mutation | GGC GGT_{biotin} ACC AGG ACA AAG GTG GGT GAG GGG ACG |
| Enzyme (SEQ ID NO: 4) | CCA AGA GTC CCC GCG GTT ACA TC_{SF}C_{SF}A/BHQ1/ |
| Photocaged Sodium | _{FAM}/T GGA TGT ATrA_{nitrobenzene} GGA AGT ACC GCC |
| Substrate (SEQ ID NO: 5) | |
| Active ATP Aptamer | T_{FAM}GU_{NH2} GU_{NH2}A CCT GGG GGA GTA TTG CGG AGG AAG |
| Amine (SEQ ID NO: 6) | GT-/Biotin/ |
| Active ATP Aptamer SF | T_{FAM}GU_{SF}GU_{SF}A CCT GGG GGA GTA TTG CGG AGG AAG |
| (SEQ ID NO: 7) | GT-/Biotin/ |
| Scrambled ATP Aptamer | T_{FAM}GU_{NH2} GU_{NH2}G ATT GGG GGA AGA ACC TGG AGC GGT |
| Amine (SEQ ID NO: 8) | TG-/Biotin/ |
| Scrambled ATP Aptamer | T_{FAM}GU_{SF}GU_{SF}G ATT GGG GGA AGA ACC TGG AGC GGT |
| SF (SEQ ID NO: 9) | TG-/Biotin/ |
| Active ATP Quencher | ACC TTC CTC CGC AAT A/iSpPC/CT CCC CCA GGT ACA |
| (SEQ ID NO: 10 and 11) | CA/3BHQ_1/ |
| Scrambled ATP Quencher | CAA CCG CTC CAG GTT C/iSpPC/T TCC CCC AAT CAC ACA |
| (SEQ ID NO: 12 and 13) | /3BHQ_1/ |
| hs.Ri.ACAA2.13.1-SEQ1 | rGrGrA rUrCrA rGrArU rArUrC rArArG rCrUrG rGrArA rGrAT |
| (SEQ ID NO: 14) | T |
| hs.Ri.ACAA2.13.1-SEQ2 | rArArU rCrUrU rCrCrA rGrCrU rUrGrA rUrArU rCrUrG rArUrC |
| (SEQ ID NO: 15) | rCrArA |

### B. Cell Lines

HepG2 cells (ATCC Product HB-8065) were cultured in DMEM (Coming) with 10% fetal bovine serum (Coming) with 100 U/mL penicillin-streptomycin (Gibco). All cells were incubated at 37° C. in 5% CO2. Once passage 30 was reached, cells were discarded.

### C. Light Irradiation

Light irradiation was performed with a Analytik (Jena, Germany) handheld UV light source. Samples were irradiated using 365 nm for 20 minutes. Sample lids were removed prior to light exposure.

### D. Synthesis

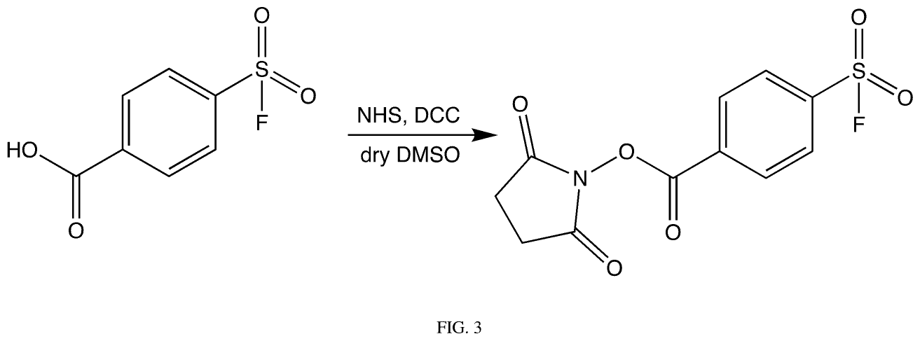

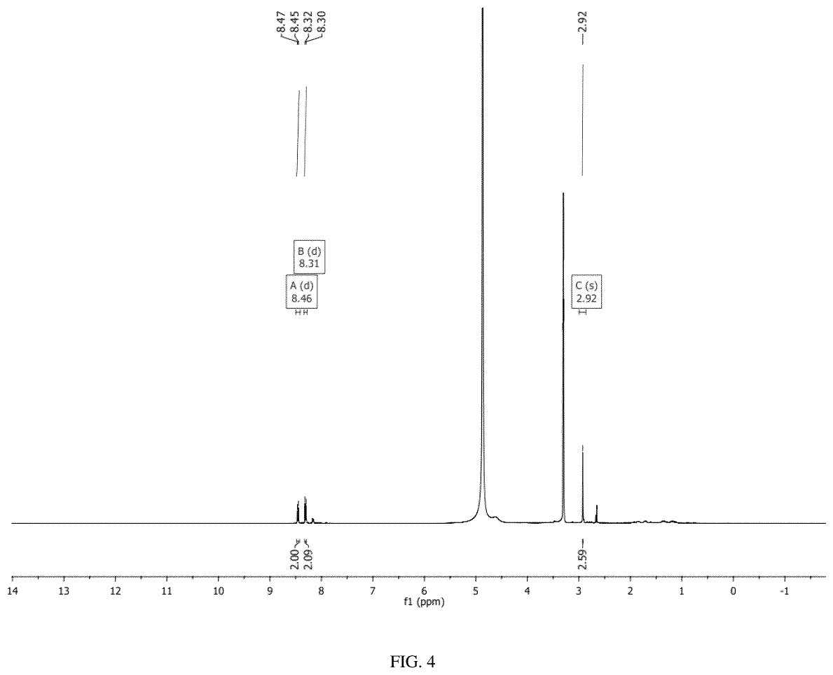

NHS-SF was synthesized as shown in FIG. 3. 4-(fluorosulfonyl)benzoic acid (0.6g, 2.8 mmol) was dissolved in 1 mL dry DMSO and 9 mL dry DCM in an oven dried glassware. N-hydroxysulfosuccinimide (0.44 g, 2.0 mmol) was added and the reaction was cooled on ice. Then dicyclohexylcarbodiimide (0.6 g, 2.8 mmol) was added and the reaction was stirred under nitrogen on ice for 4 hours. After reaction, the dicyclohexylurea side product was removed by filtration through a celite plug. The DCM was immediately evaporated and the DMSO was removed through overnight lyophilization. Product formation verified with NMR (FIG. 4). 1H NMR (400 MHZ, CD3OD) δ 8.46; (d, J=8.3 Hz, 1H), 8.31; (d, J=7.8 Hz, 1H), 2.92; (s, 2H). 1H NMR (400 MHZ, CD3OD) of (1): δ 8.46; (d, J=8.3 Hz, 1H), 8.31; (d, J=7.8 Hz, 1H), 2.92; (s, 2H).

2′-nitrobenzyl rA was synthesized in accordance with the literature (Chaulk et al., 2007).

### E. Modeling

To predict the effects of 2′ arylsulfonyl fluoride, the small molecule was manually docketed into various positions of the 8-17 DNAzyme (PDB 5XM8) in PyMOL. Distances were checked between the fluorine atom and to the opposite phosphate, adjacent phosphate, and sugar α-carbon. The 8-17 DNAzyme was used as it is a reported crystal structure, instead of a predicted structure.

### F. Secondary Structure Prediction

To predict the secondary structures of the aptamer and DNAzyme strands, the sequences were entered into the UNAFold tool through IDT. Strands were modeled as linear DNA, at 37° C. with 50 mM sodium and 5 mM magnesium. 20 maximum foldings were allowed and the most energetically favored structure reported.

### G. DNA Synthesis

Strands synthesized in house are marked in Table 1. Synthesis carried out using standard solid phase phosphoramidite chemistry on an Applied Biosciences 392 DNA/RNA synthesizer on a 1 μM scale. with Pac-dA-CEP, Pac-dG-CEP, dT-CEP, Ac-dC-CEP, 0.40 M tetrazole in acetonitrile, 5% phenoxyacetic anhydride in THF, 16% 1-methylimidazole in THF, 3% dichloroacetic acid in dichloromethane, and 0.02 M iodine in THF/pyridine/water from Glen Research. 2′ NH2 uridines were purchased from ChemGenes. For all other commercially available modifications, biotin-dT, fluorescein-dT, 5′-biotin phosphoramidite, and BHQ1-CPG were purchased from Glen Research. Modified monomers (photocaged rA base, fluorophores, biotin, and amines) were reacted for 15-minute coupling times, while all standard monomers used 25 second coupling times. After DNA synthesis, CPG beads were washed with 10% dimethylamine in acetonitrile for 5 minutes. Beads were then washed with acetonitrile and then cleaved in 80% ammonium hydroxide overnight at room temperature. Ammonium hydroxide was evaporated off with nitrogen gas. Supernatant containing DNA was separated from beads by centrifugation at 3,000 g for 5 minutes. DNA was purified either through reverse-phase HPLC using a XBridge Prep C18, 19×250 μm column with Agilent 1260 Infinity II. A gradient of 5% acetonitrile in 95% triethylammonium acetate, pH 7.0 to 60% acetonitrile in 40% triethylammonium acetate over 20 minutes was used for DMT-on purification. Samples were concentrated in butanol and concentrated to ˜500 μL. Samples were transferred to 1.5 mL Eppendorf tubes. The DMT protecting group was deprotected with neat acetic acid for 30 minutes at room temperature. DNA was desalted through overnight ethanol precipitation by adding 0.1 eq of 3 M sodium acetate and then 3 eq of ice cold 100% ethanol, vortexed, and stored at −80° C. overnight. The next day, samples were centrifuged at 15,000 g at 4° C. for 45 minutes. The supernatant was discarded and 1 mL ice cold 70% ethanol was added to the pellet. The samples were again centrifuged at 15,000 g at 4° C. for 30 minutes. The pellet was resuspended in nuclease free water and quantified by Nanodrop. All samples were stored at −80° C. after their synthesis and purification.

### H. SF Incorporation

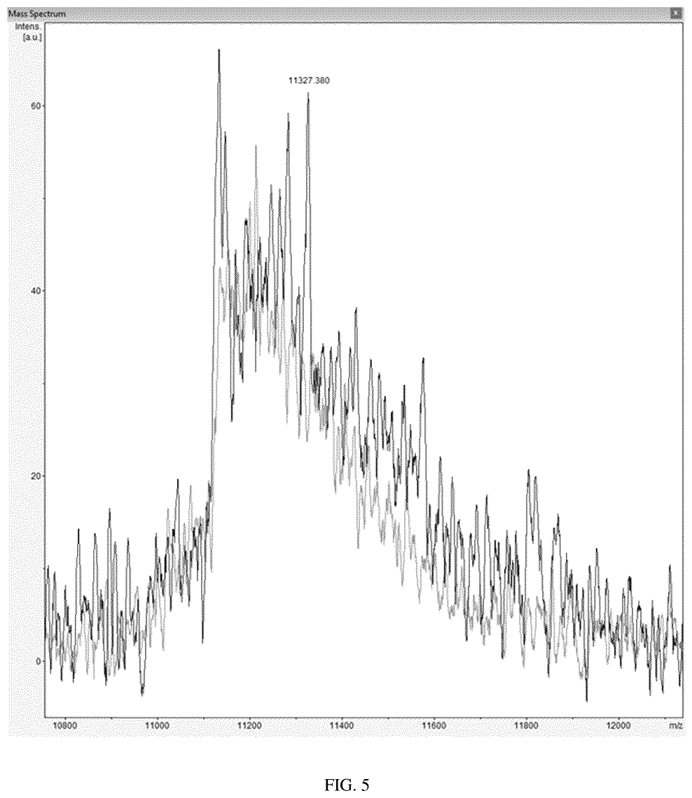

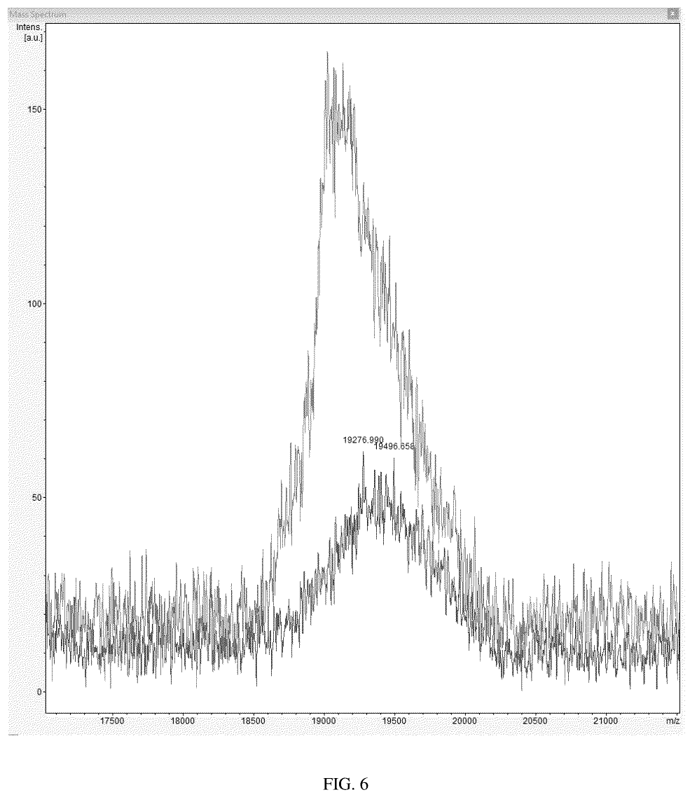

Post DNA synthesis, the small molecule sulfonyl fluoride was incorporated. Stock NH2-DNA solution (for both ATP aptamer or sodium enzyme strand) was diluted to a 10 μL solution of 300 μM NH2-DNA with 91 mM sodium tetraborate buffer, pH 8.5, in a 1.5 mL Eppendorf tube. A freshly prepared 50 mM NHS ester-SF was diluted in anhydrous DMSO. From this stock, we added 10 μL to the NH2-DNA solution in buffer. The reaction was set in a Thermomixer at 37° C. for 30 minutes. The modified DNA was then purified with a Zymo Oligo Clean and Concentrator kit following the manufacturer's protocol. The sulfonyl fluoride hydrolyzes in basic aqueous conditions, so the incorporation reaction must be performed in minimum amounts of time, and the sulfonyl fluoride modified strands need to be freshly prepared before every use. If stored, they were dried and placed at −80° C. No SF-modified strand was used more than 1 day after the modification reaction to avoid failed labeling due to SF hydrolysis. Incorporation validated with MALDI-TOF-MS (Bruker Autoflex). MALDI matrix was 3-hydroxypicolinic acid in 50:50 acetronitrile:0.1% TFA in water and 10 mg/ml diammonium hydrogen citrate. Representative mass shift in MALDI shown in FIG. 5, in which gray is the starting 2′-NH2 modified strand and blue is the post-SF incorporated strand. The instrument was operated in linear mode, which caused ions of identical mass to arrive at the detector at slightly different time points, resulting in broad peaks. Reflector mode fragmented the DNA samples due to the large electric field in the reflector.

TABLE 2
MALDI-TOF-MS of all DNA strands synthesized in house.

| | Predicted | Observed |
|---|---|---|
| Strand | Mass (m/z) | Mass (m/z) |
| Photocaged Na⁺-substrate | 7520.165 | 7427.578 |
| Active Na⁺-enzyme | 189783.6 | 19024.384 |
| Point mutation Na⁺-enzyme | 18997.6 | 19091.118 |
| Scrambled ATP aptamer | 11005.01 | 11200.870 |
| Active ATP aptamer | 11005.01 | 11133.199 |
| Scrambled ATP quencher strand with | 6914.7 | 6903.829 |
| photocaging linker | | |
| Active ATP quencher strand with | 6914.7 | 6962.043 |
| photocaging linker | | |
| Point mutation Na⁺-enzyme-SF | 19348.6 | 19344.16 |
| Active Na⁺-enzyme-SF | 19348.6 | 19276.975 |
| Scrambled ATP aptamer-SF | 11375.01 | 11322.223 |
| Active ATP aptamer-SF | 11375.01 | 11327.380 |

### I. Hybridization

For the Na+-DNAzyme sensor, 1 equivalence of enzyme to 1.2 equivalence of the photocaged substrate were diluted in 50 mM Tris, 150 mM KCl, 2 mM MgCl2, and 0.1 mM CaCl2 pH 7.4. For the ATP aptamer sensor, 1 equivalence of aptamer to 1.2 equivalence of complementary capture strand were diluted in PBS, 2 mM MgCl2. For both sensors, strands were annealed in the thermocycler by heating to 95° C. for 5 minutes, and then 60 rounds of cooling from 95° C. to 25° C. over a 30 minute period. Lower molarities of the electrophile modified strands were used to prevent unhybridized exposed electrophile.

### J. Fluorimetry

10 μM stocks were hybridized for each sensor. 1 M sodium chloride in 50 mM Bis-tris, pH 7.0, 1 mM EDTA, 10 mM sodium citrate pH 7.4 stock solution was prepared. Serial dilutions for sodium from 0 mM to 135 mM sodium were used in the 50 mM Bis-tris, pH 7.0, 1 mM EDTA, 10 mM sodium citrate pH 7.4. 2 M ATP in PBS, 2 mM MgCl2 pH 7.4 stock solution was prepared. Buffer pH after ATP addition was carefully readjusted as ATP is acidic. Serial dilutions for ATP from 0 mM to 5 mM were used in the same buffer. Final concentration of both sensors was 50 nM in 200 μL reaction volumes for fluorescence measurements in 6×50 mm borosilicate culture tubes (VWR). The ATP aptamer sensor was incubated with a range of ATP additions at 37° C. for 15 minutes before final measurements. The sodium DNAzyme was incubated with a range of sodium additions at 37° C. for 1 hour before final measurements. Initial readings were collected, with photoactivation performed on the same samples using 365 nm from a UV handheld light source for 20 minutes. Fluorescence monitored by an ISS ChronosDFD fluorometer (Urbana, IL), with excitation of 480 nm and emission monitored from 500 to 600 nm, 5 mm slit widths, and no optical density filter for all experiments.

### K. Native Gel to Evaluate Hybridization

A 5% native acrylamide gel (1 mL 10×TAE with Mg2+buffer, 1.25 mL 29:1 40% acrylamide stock, 7.75 mL water) was prepared. 10 nM of hybridized ATP aptamer sensor or 10 nM of hybridized sodium-DNAzyme sensor stocks were diluted with 1.2 μL 10×TAE with Mg2+, 2 μL 6×gel loading dye (purple) (BioRad), and 7.8 μL of water. The gel was precooled at 4° C. and then samples were directly loaded into the gel at 4° C. The gel was run at 120 V for 2 hours in TAE+2 mM MgCl2 at 4° C. Gels were directly visualized in the gel imager using the fluorescein on the DNA strands. Complementary strands without quencher were used for these assays as the quencher overpowered the sensor fluorophore and SYBR Gold staining.

### L. Electrophoretic Mobility Shift Assay for In Vitro Protein Labeling

30 82 M of DNA aptamer or Na+-DNAzyme enzyme was hybridized with 45 μM of capture or substrate strand, respectively, in low ionic strength hybridization buffer (10 mM HEPES and 20 mM MgCl2) following the hybridization protocol. The hybridized samples were decaged with 365 nm for 20 minutes using a Analytik (Jena, Germany) handheld UV light source. For the ATP aptamer, 0.8 mM ATP was added to the samples and incubated for 15 minutes at room temperature. Samples were then transferred into a 0.5 mL 3K molecular weight cut off (Amicon) pre-wetted filter with the addition of 400 μL of the low ionic strength hybridization buffer. The samples were filtered centrifuged 3× for 15 minutes at 15000 g. This step was imperative as it removed excess or reacted capture/target strand. If not removed, the excess DNA served as a blocking agent to inhibit the SF-mediated protein tagging. Samples were collected and placed into the speed vacuum until a 20 μL volume was reached. Next, 1.6 μM of protein was added (thrombin) into the samples and the reaction was allowed to proceed over the course of 16 hours in a thermomixer at 37° C. Stop solution (4×Laemmli protein sample buffer, Bio-Rad) was added and samples where heated to 90° C. for 15 minutes. Samples were loaded onto a SDS 4-20% gradient pre-casted gel (Bio-Rad) and the gel was run for 70 minutes at 120 V. Once complete, the gel was washed with ultra-pure water for 3× before staining with SYPRO-orange (Thermo-Fisher Scientific) per the manufacturer instructions. The stained gel was then imaged using Biorad Gel Doc XR+ Imaging System.

### M. Cell Delivery for Proximity Labeling

HepG2 cells were plated in ibidi 8 well plates (for imaging), Corning 6 well plates (for Western blots), or Fisher 10 cm plates (for quantitative proteomics) the day before the experiment to prevent overclumping of cells. All plates used were pre-TC treated, individually wrapped, and sterilized by the manufacturer. 400 nM of annealed sensor was transfected into cells with Turbofect and serum-free DMEM, following the manufacturer's instructions. Cells were incubated with the probe for 4 hours in the cell culture incubator. Before light activation, the media was replaced with fresh DMEM (10% FBS, 100 U/mL penstrep, Corning). Light activation was performed for 20 minutes at 365 nm. The cells were then incubated for an additional 4 hours in the incubator before downstream sample preparation for respective experiments.

### N. Cell Delivery for siRNA

HepG2 cells were plated 2 days before cells were needed for imaging or western blot experiments. Negative control DS NCI and target siRNA were bought predesigned from IDT. 30 nM of each siRNA were delivered with Turbofect, following the manufacturer's protocol. After 6 hours, the delivery media was replaced with fresh DMEM (10% FBS, 100 U Pen/Strep). The cells were allowed to grow for 2 days after siRNA delivery.

### O. Confocal Imaging

Following the cell delivery for proximity labeling protocol, cells were washed 3× with PBS, fixed with 4% paraformaldehyde in PBS (Fisher) for 10 minutes at room temperature, washed 3× with PBS, permeabilized with 0.2% Triton X-100 in PBS for 10 minutes at room temperature, washed 3X with PBS, and then blocked with 1% BSA+0.3 M glycine in PBS for overnight at 4° C. We found the glycine addition was necessary to minimize background autofluorescence, as noted in our no DNA controls. Then 2 μg/mL Cy5-streptavidin (Sigma) was added in fresh Opti-MEM to the cell culture per well for 45 minutes at 37° C. The cells were washed with PBS and the media was replaced with fresh PBS prior to imaging. Images were collected on a Zeiss 710 confocal microscope with 63× oil magnification. Lasers 488 (collect 500 to 540), 543 (collect 560 to 630), and 633 (collect 650 to 750) were used with frame collections and pinhole size set to 1 Airy Unit. Images were focused using brightfield, as laser intensity during focusing significantly decreased Cy5 signal. Images were also collected only in the center of each well, due to fluorescence intensity distortion at well edges. Three biological replicates were performed for each sensor. The fluorescence intensities were quantified in Fiji. The fluorescence channel of interest was overlaid on each respective brightfield channel. Intracellular fluorescence was based on the mean of the overlaid fluorescence corresponding to the intracellular environments. Statistical analysis was performed with a 2-tailed student's t-test, in which data is represented as the mean fluorescence with errors as standard deviation.

### P. Western Blots with Cell Lysates

Following the cell delivery for proximity labeling protocol, cells were washed 1× with ice cold PBS. 250 μL of ice-cold RIPA lysis buffer with freshly diluted 1×Halt phosphatase and protease inhibitor cocktail (Thermo Fisher) was directly added to the each well. The cell culture plates were incubated on ice for 5 minutes. Cells were scraped and transferred to a 1.5 mL low-bind Eppendorf tube. Total protein concentration was quantified with Pierce BCA assay (Thermo Fisher following the 30 minutes, 37° C. protocol). 10 μg of total protein from crude lysed cells was heated to 60° C. for 15 minutes with 4×Laemmli loading dye and 100 mM DTT. The samples were then run on a 4-20% precast SDS-PAGE gel at 120 V for 1 hour in homemade Tris-HCl glycine running buffer. One sheet of extra thick filter paper was soaked in cathode running buffer (1×Tris/CAPS, 0.1% SDS), with buffer made from 10×Tris/CAPS (Bio-Rad) for 10 minutes at 4° C. One sheet of extra thick filter paper was soaked in anode running buffer (1×Tris/CAPS, 15% methanol), with diluted buffer made from 10X Tris/CAPS (Bio-Rad) for 10 minutes at 4° C. The PVDF, low fluorescence, 0.45 μm, 7×8.5 cm precut membrane was prewet with methanol for 30 seconds until transparent and then equilibrated in anode buffer for 5 minutes. The gel was washed with water three times and incubated at 4° C. for 10 minutes in the cathode buffer. The transfer sandwich was prepared in a Bio-Rad semidry transfer system and run at 15 V for 15 minutes. The membrane was then dried at 37° C. for 10 minutes. Afterwards, it was rehydrated in 100% methanol for 30 seconds, washed with ultrapure water, and immersed in Revert 700 Total Protein Stain (Licor) for 5 minutes. Excess stain was decanted. The membrane was washed 2× with 6.7:30:63.3 AcOH:MeOH:H2O for 30 seconds each and placed in ultrapure water. The total protein stain was imaged using a ChemiDoc Imaging Station (Bio-Rad) in the 680 nm channel. The membrane was blocked with Licor Intercept blocking buffer at room temperature for 1 hour, washed 3× with TBST (5 minutes each at room temperature), then incubated with the stain of interest. For proximity labeling, we used a 1:1000 dilution of Streptavidin IR800 (Licor P/N 926-32230) for 30 minutes at room temperature. For siRNA knockdown, APOA2 (1:5000, Invitrogen PAI-26900) was incubated for 1.5 hours at room temperature, AHSG (1:5000, OriGene OTI2H2) was incubated at room temperature for 1.5 hours, ACAA2 (1:2500, Invitrogen PA5-59942) was incubated at room temperature for 1.5 hours, and APOE (1:2500, Invitrogen 16H22L18) was incubated at room temperature for 1.5 hours. Secondary antibodies used were donkey anti-goat Alexa Fluor 647 (Invitrogen A-21447) and goat anti-rabbit Alexa Fluor 555 (Invitrogen A-21428), all at 1:1000 dilutions, incubated for 1.5 hours at room temperature. All dilutions were performed in Licor Intercept blocking buffer. After primary antibody, the membrane was washed 3× with TBST (5 min cach). The membrane was incubated in TBS for 5 min and imaged with a ChemiDoc Imaging Station (Bio-Rad). Three biological replicates were repeated.

### Q. Pulldown with Streptavidin Magnetic Beads

Following the cell delivery for proximity labeling protocol, cells were washed 1× with ice cold PBS. Ice cold RIPA lysis buffer (with freshly added 1×Halt phosphatase and protease inhibitor) was directly added to the cell culture dish (250 μL per well) and incubated on ice for 5 minutes. Cells were scraped and transferred to an Eppendorf tube. Total protein concentration was quantified with BCA assay (30 minutes, 37° C. protocol). For pull-down, 250 μL Dynabeads MyOne Streptavidin C1 were prewashed twice with 1 mL RIPA buffer. 500 μg of protein was transferred to beads. Samples were vortexed until homogenous (˜10 seconds) and incubated at 4C overnight with end over end rotation. Beads were then pelleted and washed 3× with 1 mL 1% SDS in PBS, 3× with 1 mL 1 M NaCl in PBS, and 3× with freshly prepared 1 mL 10% EtOH in PBS. For each wash, the beads were thoroughly resuspended in washing solution before pelleting (vortex ˜10 seconds). The beads were then reconstituted in 500 μL of RIPA buffer, with the 500 μL split between 2 new LoBind Eppendorf tubes. One tube went through 35 μL of Elution 1 buffer (10 mM EDTA, 95% formamide, pH 8.2) and the second tube went through 40 μL of Elution 2 buffer (6 M urea, 30 mM biotin, 2% SDS in PBS, 25% Laemmli loading dye, 5 mM DTT). Both tubes were heated for 15 min at 60° C. Then 4×Laemmli loading dye+5 mM DTT was added to the Elution 1 tube. The beads were pelleted and the whole elution buffer was loaded onto a 4-20% SDS-PAGE gel. The general western blot procedure was then followed. Three biological replicates were repeated.

### R. General Proteomics Procedure

Following the cell delivery for proximity labeling protocol, cells were washed 1× with ice cold PBS. Ice cold RIPA lysis buffer (freshly added 1×Halt phosphatase and protease inhibitor) was directly added to the cell culture dish (1 mL per well) and incubated on ice for 5 minutes. Cells were scraped and transferred to an Eppendorf tube. Total protein concentration was quantified with BCA assay (30 minutes, 37° C. protocol). For proteomics, 250 μL Dynabeads MyOne Streptavidin Cl were prewashed twice with 1 mL RIPA buffer. Per biological replicate (total of 3 per sample), 1.5 mg of total protein was transferred to beads. After vortexing for 10 seconds, the samples were incubated overnight on a rotisserie at 4° C. The beads were pelleted and washed 3× with 1 mL 1% SDS in PBS, 3× with 1 mL 1 M NaCl in PBS, and 3× with freshly prepared 1 mL 10% EtOH in PBS. For cach wash, the beads were thoroughly resuspended in washing solution before pelleting (vortex ˜10 seconds). The beads were then transferred to a new LoBind Eppendorf tube and washed 3× with 0.5 mL PBS and 3× with 0.5 mL 100 mM ammonium bicarbonate in water. The beads were resuspended in 500 μL 3 M urea in PBS. Then 25 μL of freshly prepared 200 mM DTT in 25 mM ammonium bicarbonate was added and incubated at 55° C. for 30 minutes with end over end rotation. In the dark, a freshly prepared 30 μL solution of 500 mM iodoacetamide in 25 mM ammonium bicarbonate was added and incubated for 30 minutes at room temperature with end over end rotation. The supernatant was removed and the beads were washed 3× with 0.5 mL PBS and 3× with 0.5 mL 50 mM tricthyl ammonium bicarbonate (TEAB). The beads were resuspended in 0.5 mL 50 mM TEAB and transferred to a new LoBind tube. The beads were resuspended in 40 μL 50 mM TEAB and 2 μL of trypsin (Pierce mass spectrometry grade). The tubes were rotated overnight at 37° C. with end over end rotation. After 16 hours, 1.0 μL trypsin was added and the beads rotated another 1 hour at 37° C. TMT 6-plex (0.8 mg) were warmed to room temperature before opening the package. Right before bead pelleting, the TMT labeling reagents were diluted with 41 μL of anhydrous acetonitrile and thoroughly vortexed. The supernatant was directly added to the appropriate TMT 6-plex tube and incubated for 2 hours at room temperature with a Thermomixer. The samples were quenched with 8 μL of freshly prepared 5% hydroxylamine in water and incubated at room temperature in the Thermomixer for 15minutes. The samples were pooled in a single LoBind tube and quenched with 16 μL TFA (optima grade). The peptides were dried to completion in a SpeedVac (45° C.) and redissolved in 300 μL of 0.1% TFA in water. The pooled peptides were fractionated using the Pierce High pH Reverse Phase Peptide Fractionation Kit and subsequently repooled. Sample I pooled fractions 1, 4, and 7. Sample 2 pooled fractions 2 and 6. Sample 3 pooled fractions 3, 5, and 8. The samples were dried to completion in a SpeedVac (45° C.). Samples were run with Ultimate 3000 RSLCnano UPLC, 25 cm×75 μM column with a 300 nL/min flow rate and 120 minute gradient on a Thermo Orbitrap Fusion hybrid mass spectrometer. SPS-MS3 was used. MS/MS/MS data was searched against the Uniprot human protein database. Raw files were converted to mzML using Fragpipe with suggested standard settings. Peak picking and zero samples set as filters. MSFragger2 was used for database searching, with variable modifications set to methionine oxidation and N-terminal acetylation and deamidation. Fixed modifications were set to cysteine carbamidomethylation, with a maximum of 5 modifications per peptide. Tryptic cleavage was selected with a maximum of 2 missed cleavages. The maximum peptide mass was set to 6000 Da. The label minimum ratio count was set to 2 and quantified using unique and razor peptides. FTMS MS/MS match tolerance was set to 0.05 Da, and ITMS MS/MS match tolerance was set to 0.6 Da. All other settings were left as default. The proteinGroups.txt file was imported into Perseus (https://maxquant.net/perseus/). The data were subsequently filtered based upon the following criteria, ‘only identified by site’, ‘reverse’, and ‘potential contaminant’. The resulting data was Log2 transformed, and median normalization was performed. FDR-corrected p-values were determined by a 2-sample T-test after Benjamini-Hochberg procedure. Proteins with only 1 identification were removed from the data set. The data were visualized by plotting as a volcano plot in GraphPad Prism.

### S. Fluorescence Recovery after Photobleaching (FRAP) to Measure Diffusion Coefficient

A test DNA sequence (5′ FAM-GGC GGT ACC AGG TCA AAG GTG GGT GAG GGG ACG CCA AGA GTC CCC GCG GTT ACA TCC A 3′) (SEQ ID NO: 18) was delivered into HepG2 cells following the cell delivery protocol. The delivery occurred over 4 hours in the cell culture incubator at 37° C. Prior to imaging, the cells were washed with PBS and replaced with fresh DMEM, serum free. Cells were imaged on a Zeiss 710 confocal microscope, with a 40× water immersion objective for a starting point image. Three regions of interest were applied with equal size circular regions for all three spots. A sample of interest, a similar fluorescent intensity reference sample, and a background in a location with no cells were used. The FRAP module in Zen was used to photobleach the sample of interest with 100% 488 nm laser, with image collections at 1 image per minute for 25 minutes. The data were fit with the standard exponential fitting [b(1−exp(−x*tm))+c] to obtain the mobile and immobile fractions and half-life for recovery (T1/2). From this data, the diffusion coefficient was calculated using D=0.25*radius (bleach laser)2/T1/2 3. The radius of the uniform bleach laser was 0.22 microns. This resulted in an estimated diffusion coefficient of 0.000018267 microns2/second or 0.001096 microns2/minute. Three biological replicates were repeated.

### T. Sodium Green Imaging

HepG2 cells were plated in an ibidi 8 well plate 2 days before imaging. siRNA was delivered following the general siRNA delivery protocol. After 2 days, a 2 mM stock of Sodium Green Tetraacetate (ThermoFisher) was freshly prepared in DMSO. The stain was diluted to 10 μM per well in DMEM, serum free (Corning) to prevent early hydrolysis. The diluted stain was incubated in cach well for 1 hour at room temperature. Cells were washed once, and media was replaced with PBS before imaging. Three biological replicates were repeated. The fluorescence intensities were quantified in Fiji. The fluorescence channel of interest was overlaid on cach respective brightfield channel. Intracellular fluorescence was based on the mean of the overlaid fluorescence corresponding to the intracellular environments. Statistical analysis was performed with a 2-tailed student's t-test, in which data is represented as the mean fluorescence with errors as standard deviation.

### U. Immunofluorescence Staining

HepG2 cells were plated in an ibidi 8 well plate 4 days before imaging. On the second day, siRNA was delivered following the generalized siRNA delivery protocol. On the third day, the modified DNAzyme system was delivered following the cell delivery for proximity labeling protocol. After 8 hours, the cells were washed 3× with PBS, fixed with 4% paraformaldehyde in PBS (Fisher) for 10 minutes at room temperature, washed 3× with PBS, permeabilized with 0.2% Triton X-100 in PBS for 10 minutes at room temperature, washed 3× with PBS, and then blocked with 1% BSA and 0.3 M glycine in PBS for overnight at 4° C. On the 4th day, each well was incubated with its respective primary antibody. APOA2 (1:100, Invitrogen, RRID: AB_2532435) was incubated for 1 hour at room temperature, AHSG (1:100, OriGene, RRID: AB_2622514) was incubated at room temperature for 1 hour, ACAA2 (1:100, Invitrogen, RRID: AB_2637576) was incubated at room temperature for 1 hour, and APOE (1:100, Invitrogen, RRID: AB_2532438) was incubated at room temperature for 1 hour. Cells were washed 3× with PBS and then incubated with the fluorophore-conjugated secondary antibody. Secondary antibodies used were donkey anti-goat Alexa Fluor 555 (Invitrogen, RRID: AB_2535853) and goat anti-rabbit Alexa Fluor 555 (Invitrogen, RRID: AB_2525849), all at 1:100 dilutions and incubated for 1 hour at 37° C. Simultaneously with the secondary antibodies, 2 μg/mL Cy5-streptavidin was incubated in each well to stain the labeled proteins. All antibody dilutions were all made in 1% BSA+0.3 M glycine in PBS blocking buffer. Negative controls of no primary antibody were used to gate for background non-specific antibody signal. Prior to imaging, cells were washed 1× and replaced with fresh PBS. Three biological replicates were repeated.

### V. MTT Assay

HepG2 cells were seeded in a 96 well plate at 90% confluency. For experimental samples, 400 nM SF-modified ATP aptamer was delivered with Turbofect for 4 hours and 400 nM SF-modified ATP aptamer delivered with Turbofect+365 nm light activation were used. As a positive control, 30% hydrogen peroxide was added. As a negative control, only HepG2 cells were used. After the 4 hour incubations at 37° C., a solution of MTT (stock 5 mg/mL in PBS) was diluted in cell culture media to a final concentration of 0.25 mg/mL. The cell culture media was removed and replaced with 100 μL of the diluted MTT. The plates were incubated at 37° C. for 2 hours. The MTT solution was then aspirated and replaced with 200 μL DMSO to solubilize the purple formazan product. Cell viability was assayed by measuring absorbance at 570 nm on Agilent BioTek Synergy H1 plate reader. Three technical replicates were repeated.

### W. ACAA2 Kinetics

The starting reaction mixture contained 50 mM Tris-HCl at pH 8.1, 2 mM MgCl2, 60 μM coenzyme A (hydrate), and 80 μM acetoacetyl-CoA. Either no additional salt was added or a range of 5 to 20 mM sodium chloride or potassium chloride was added to the starting reaction mixture. UV-Vis spectroscopy was acquired on an Agilent 8453 spectrometer. 0.5 μg ACAA2 (1 μL, Abcam) was quickly added to the cuvette to initiate the enzymatic reaction. Reaction kinetics were monitored every 5 seconds at 303 nm at 25° C. with a rice stir bar in a quartz cuvette with 1 cm path length over 10 minutes. Initial readings were taken before addition of enzyme after the first reading. Each sample was blanked with the Tris-HCl buffer. Data was baseline subtracted with absorbance readings averaging over 600 nm to 800 nm for the entire 10 minute measurements. Data was normalized to the highest absorbance reading after the removal of the pipette from the cuvette during enzyme addition (the absorbance at the 2nd 5 second reading was set to the maximum). Data was processed using KinTek Explorer. Three biological replicates were repeated.

### Example 2—Proximity Labeling of Proteins Near ATP Using an ATP-Responsive Aptamer

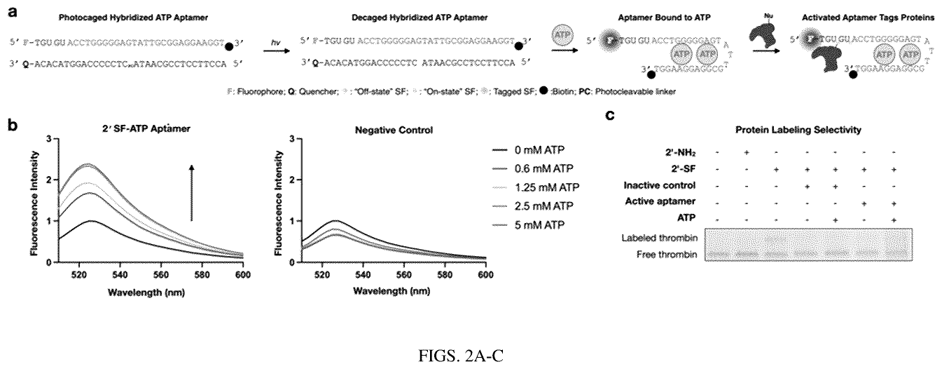

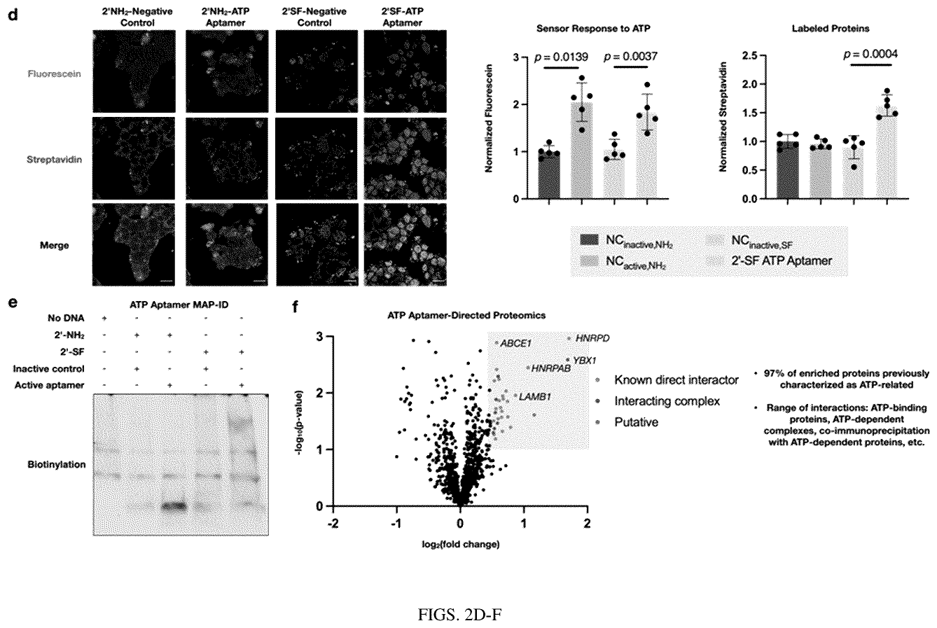

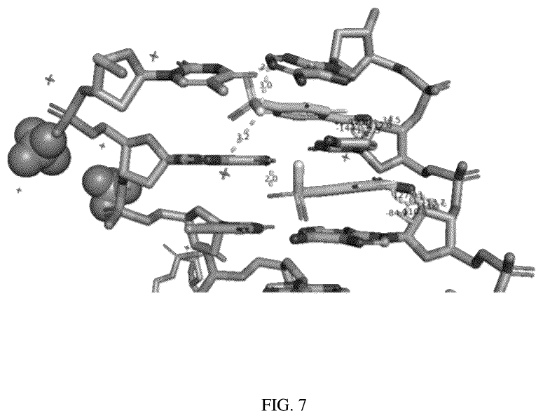

To identify proteins in proximity of a specific metabolite, ATP was selected and its corresponding ATP-responsive aptamer to develop and demonstrate DAP-ID. This aptamer was chosen for its well-characterized structure upon binding ATP-containing small molecules and wide use in the literature. Specifically, the conserved ATP-binding sequence 5 was used (FIG. 2 panel A, shown in purple) and the 5′ binding arm was extended. In this extended region, two uridines were modified with an aryl sulfonyl fluoride (SF) group at the sugar's 2′-carbon (FIG. 2 panel A). To protect the SF group from reacting with off-target proteins before the aptamer binds ATP, a complementary strand spanning both the conserved ATP-binding region and the extended region was hybridized. Molecular simulation suggests that the SF group's aryl ring can form π-π stacking interactions with the nucleotides in the complementary strand, sterically shielding the electrophile within the sensor's duplex (FIG. 7). Furthermore, to allow spatiotemporal control, a photocleavable (PC) o-nitrobenzyl group was introduced to the complementary strand (Hong et al., 2020). After delivering the probe into HepG2 cells, the probe was photoactivated to cleave the PC group and split the complementary strand into two distinct strands. This decreased the predicted melting temperature between the complementary strand and the aptamer. The weakened hybridization enabled the aptamer strand to bind ATP, dehybridize, and expose the 2′-SF for nearby protein accessibility (FIG. 2 panel A). To visualize the sensor's response to ATP, the aptamer and complementary strand were modified with a fluorophore and quencher, in which ATP-specific dehybridization results in fluorescence increase. A 3′ biotin was also installed on the aptamer as a handle for tagged protein identification.

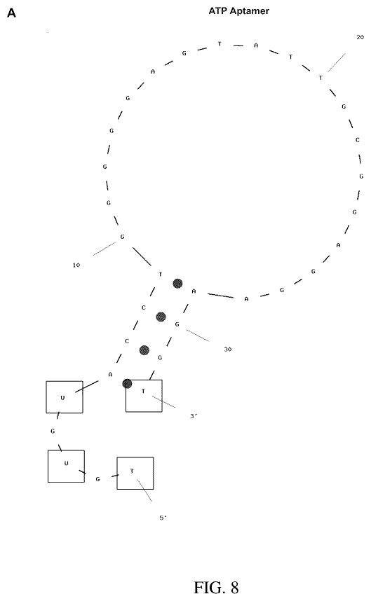

Even though the above modifications are distal from the ATP aptamer's binding pocket based on its predicted secondary structure (FIG. 8), the modifications did not compromise the aptamer's ability to detect ATP. Using fluorescence spectroscopy, it was found that the modified ATP aptamer sensor displayed nearly 3-fold fluorescence increase in response to 5 mM ATP—the average intracellular level of ATP (FIG. 2 panel B). To ensure that the observed fluorescence signal was ATP-specific, a negative control (NCinactive) was designed, in which the ATP aptamer's sequence was scrambled, while keeping its predicted hybridization temperature with the complementary strand above 45° C. to limit background dehybridization. This NCinactive displayed negligible fluorescence increase in response to ATP, confirming the active aptamer's sensitivity and activity toward ATP detection (FIG. 2 panel B).

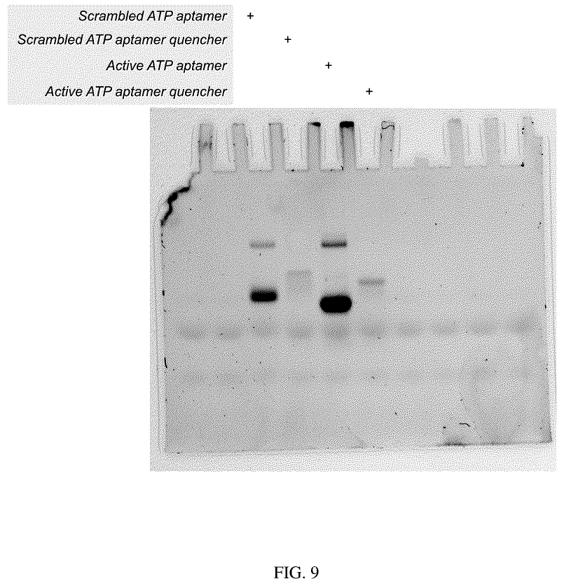

To investigate DAP-ID's protein labeling selectivity and efficiency in vitro, the modified ATP aptamer was used to label the model protein thrombin. The ATP aptamer's G-quadruplex binds thrombin non-covalently (Zhang et al., 2021), placing the aptamer in close proximity to thrombin and mimicking the SF-mediated proximity-enhanced reaction in cellular environments. Three controls were used: 1) an unhybridized 2′-NH2 in place of the 2′-SF to account for any non-covalent interactions; 2) an unhybridized 2′-SF as a positive control to account for the maximum labeling yield; and 3) the hybridized NCinactive,SF with the 2′-SF modification but a scrambled sequence to account for background labeling by SF devoid of ATP binding. The labeling efficiency and selectivity through electrophoretic mobility shift assays were assessed. The pre-exposed 2′-NH2 did not result in detectable protein labeling, indicating removal of all non-covalent interactions (FIG. 2 panel C). Conversely, the pre-exposed 2′-SF resulted in a major labeled protein band shift, with a maximum labeling efficiency of 30% as a positive control (FIG. 2 panel C). This labeling efficiency is similar to literature reports of other aptamer systems modified with SF groups (Shi et al., 2025). The NCinactive,SF did not result in detectable protein labeling, either in the absence or presence of ATP. Likewise, the SF-modified ATP aptamer without ATP did not result in labeled protein, indicating the electrophile was successfully hidden within the sensor's duplex. Only the 2′-SF active ATP sensor with the addition of ATP achieved detectable protein labeling with similar labeling efficiency (5%) as the positive control of the 2′-SF pre-exposed (FIG. 2 panel C). To verify that this result was not an artifact of incomplete sensor hybridization, complete sensor hybridization was confirmed with a native gel (FIG. 9).

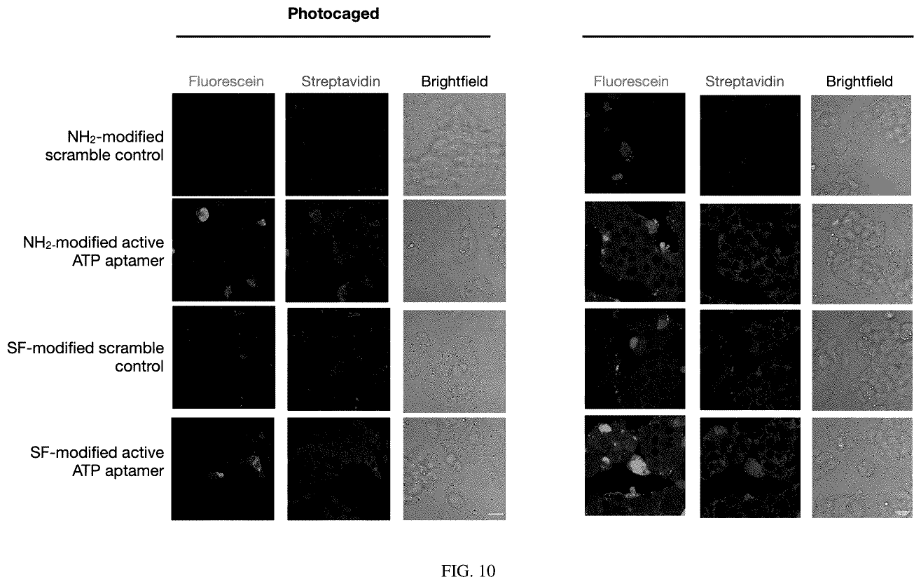

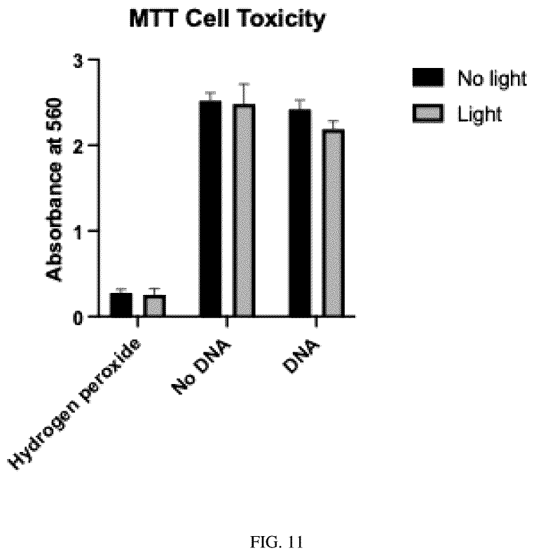

After demonstrating that DAP-ID can label proximal proteins in vitro, the platform in HepG2 cells after delivery, photoactivation, and intracellular response was imaged. To account for background labeling, we three controls were used: 1) hybridized NCinactive,NH2, 2) hybridized NCactive,NH2, and 3) hybridized NCinactive,SF. Both NCactive,NH2 and the 2′-SF ATP aptamer displayed a ˜2-fold increase in fluorescein signal compared to their respective negative controls, indicating that neither modification affected the ability of the active probe to detect ATP. On the other hand, a 1.8-fold increase was observed in streptavidin signal with the 2′-SF active ATP aptamer compared to the NCinactive,NH2 control, indicating labeled proteins (FIG. 2 panel D). All groups without light activation showed minimal fluorescence response (FIG. 10). To verify that fluorescence signal increases were not a result of cellular stress due to the photoactivation or DNA delivery, a cytotoxicity assay was performed and it was found the experimental protocols had minimal effect on cell viability (FIG. 11).

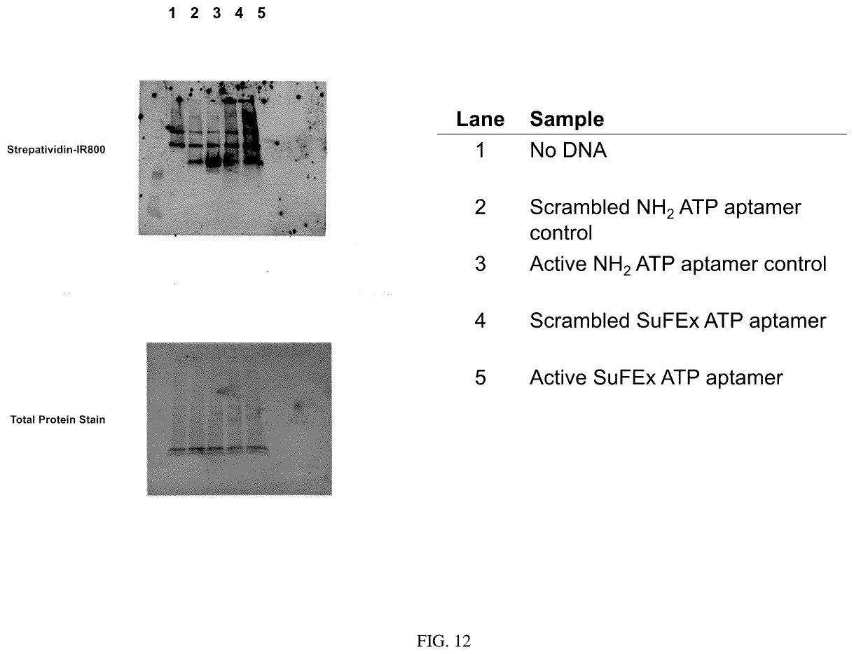

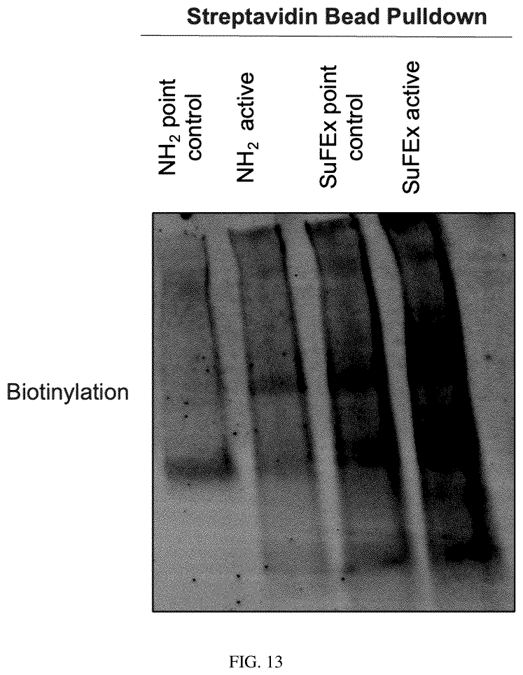

To further determine the efficiency of protein labeling the DAP-ID platform was delivered into intact HepG2 cells and ran a Western blot on the cell lysate. Increased biotinylation from the 2′-SF ATP aptamer was found, indicating successful ATP aptamer-guided protein biotinylation (FIG. 2 panel E), while total protein staining demonstrated equal loading per lane (FIG. 12). Moreover, the NCinactive,NH2, NCactive,NH2, and NCinactive,SF controls did not show significant labeling above background biotin-dependent carboxylases (Cho et al., 2020). No significant distinct bands were observed, suggesting a low labeling efficiency of a wide range of proteins. To verify that DAP-ID covalently labeled proteins, the biotinylated species was purified using streptavidin magnetic beads. A Western blot with streptavidin staining confirmed significant enrichment of the active SF-modified ATP aptamer compared to the NCinactive,NH2, NCactive,NH2, and NCinactive,SF controls (FIG. 13).

To identify proteins that have been labeled by DAP-ID, quantitative mass spectrometry through tandem mass tag (TMT) isobaric labeling was used. As the negative control to account for off-target labeling due to sensor degradation, dehybridization, or nonspecific interactions with proteins in the absence of ATP, the NCinactive,SF control was used. Three biological replicates with each group were performed, using 1 mg of total protein input, streptavidin bead purification, and on-bead sample preparation. Quantitative proteomics for the active ATP aptamer revealed 29 enriched proteins with log2(fold change)>0.5 and p-value<0.05, after Benjamini-Hochberg correction (FIG. 2 panel F) (Benjamini et al., 1995). Of these enriched proteins, 97% are direct interactors with ATP (shown in gold) or direct interactors of ATP-regulated proteins (shown in blue) (FIG. 2 panel F, Table 1). For example, DAP-ID enriched several heterogenous nuclear ribonucleoprotein components of the ATP-dependent spliceosome complexes, which are allosterically activated to perform ATP hydrolysis (Kim et al., 1993). Other ATP-related proteins included Y-box binding protein, which localizes with ATP-dependent DDX1 (Onishi et al., 2008), ATPase nucleolin (Miranda et al., 1995), RuvB-like 1 ATPase (Puri et al., 2007), ATPase kinesins (Miki et al., 2001), and ATP-binding serine/threonine kinases (Hanks et al., 1988). Interactors of ATP-dependent proteins included cingulin, which is directly linked to ATP-dependent myosin (Rouaud et al., 2023), APOBEC-3B, which interacts with ATP-dependent hnRNPs (Hein et al., 2015), SAFB-like transcription modulator, which interacts with ATP-dependent hnRNPs (Nayler et al., 1998), and laminin, which interacts with ATP-allosterically stabilized integrin (Martin et al., 2022). Compared to the whole protcome of HepG2,52 our identified proteins were not simply the most highly expressed proteins in the cell.

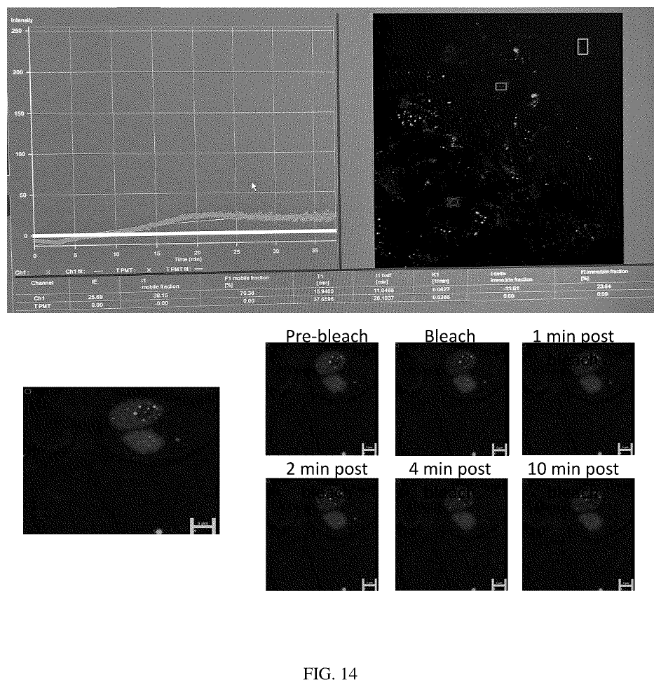

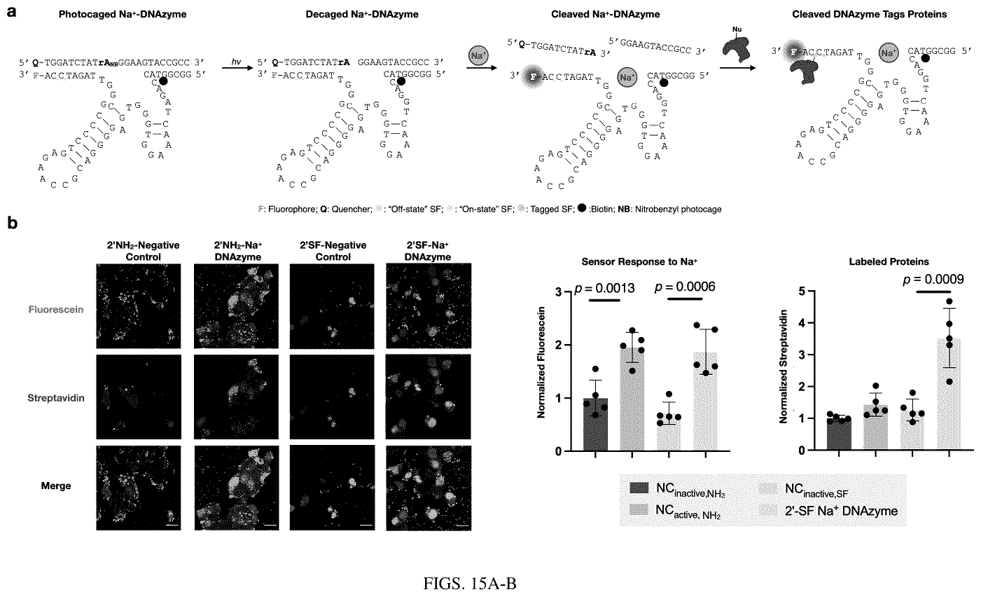

To determine DAP-ID's labeling resolution, the platform's diffusion coefficient was measured. While the literature supports the slow diffusion of large macromolecules (Lukacs et al., 2000), the system's specific diffusion was measured quantitatively. Through fluorescence recovery after photobleaching, the diffusion coefficient was determined to be ˜1.83×10-9 cm²/s, corresponding to a diffusion of 3×10-5 cm² as an approximate measure of DAP-ID's diffusion (FIG. 14). Other cytoplasmic diffusion measures of similarly sized nucleic acids were on the order of 10-8 cm²/s.54 Without wishing to be bound by any theory, based on the combination of the low sensor diffusion with the SF's close proximity requirements for protein labeling (Yang et al., 2018; Luy et al., 2020), the inventors hypothesized non-proximal, off-target protein labeling distal from the site of sensor activation is low.

### Example 3—Demonstration of the System's Generalizability with a Na⁺-DNAzyme

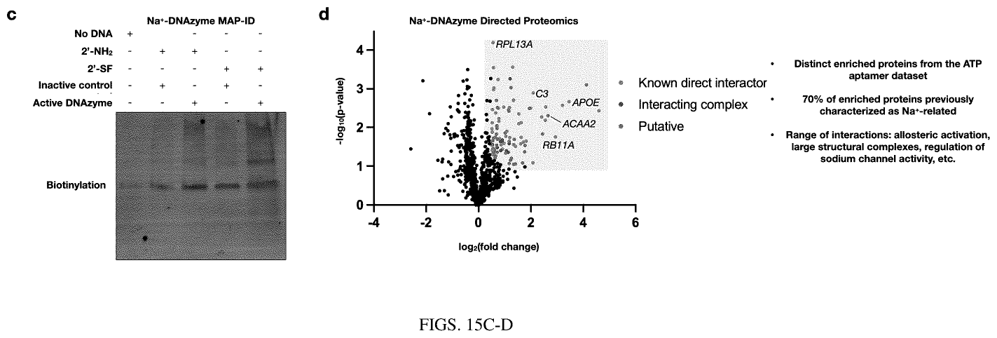

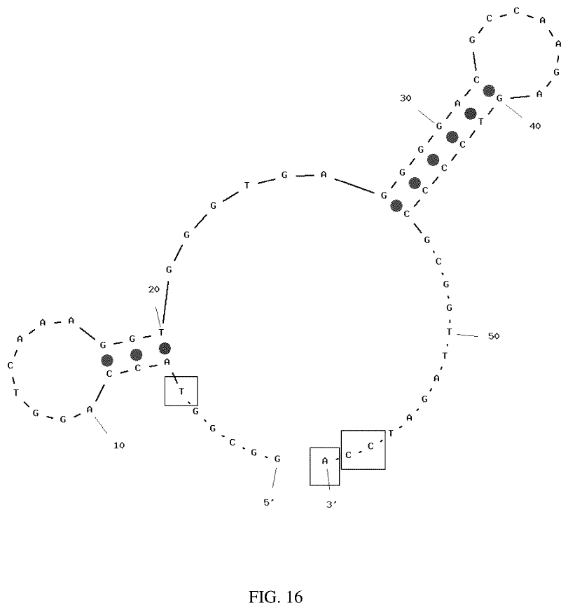

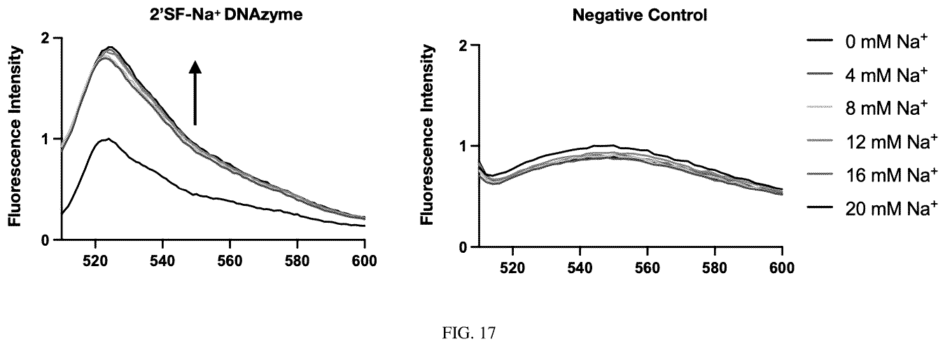

To test the system's generalizability and to further rule out any artifacts that the direct interaction between the DNA aptamer and the proteins in the absence of metabolites or metal ions play any role in the findings, DAP-ID was applied to detect proteins in proximity of metal ion pools using a Na+-selective DNAzyme, which cleaves its substrate in response to intracellular levels of sodium (Torabi et al., 2015). Select 2′-SF modifications were placed in the DNAzyme's binding arm. To hide the SF groups prior to sensor activation, the DNAzyme was hybridized with its complementary substrate strand. For spatiotemporal control, the 2′-OH of the adenosine ribonucleotide (rA) at the scissile position was photocaged with an o-nitrobenzyl PC group (Torabi et al., 2015). After cellular delivery, the DNAzyme was photoactivated to restore its active state, allowing for substrate strand cleavage induced by Na+. The substrate strand cleavage reduced the duplex melting temperature, weakening the hybridization of the binding arms between the DNAzyme and substrate strand, and ultimately unveiling the SF. To visualize the sensor in cells, the DNAzyme was modified with a fluorescein and the substrate with a corresponding quencher. For protein identification, a biotin was installed within the DNAzyme (FIG. 15 panel A). As a negative control, a single-point mutation in the DNAzyme (NCinactive) was used to abolish any Na+-specific activity (Torabi et al., 2015). Although modification positions were distal from the DNAzyme active site (FIG. 16), the sensor's response was tested using fluorescence spectroscopy to confirm sodium-specific cleavage. The active Na+-DNAzyme, but not the inactive point mutation control, displayed a fluorescence increase in response to intracellular levels of Na+ (FIG. 17).

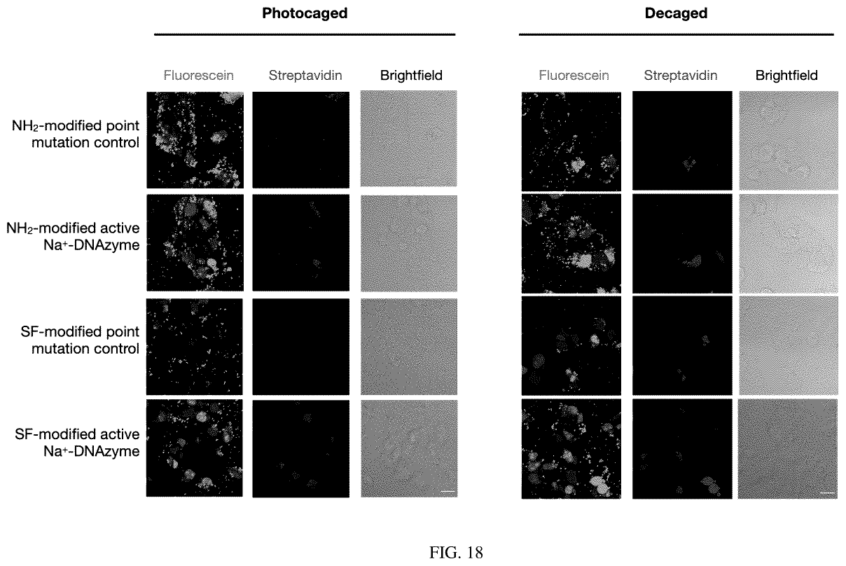

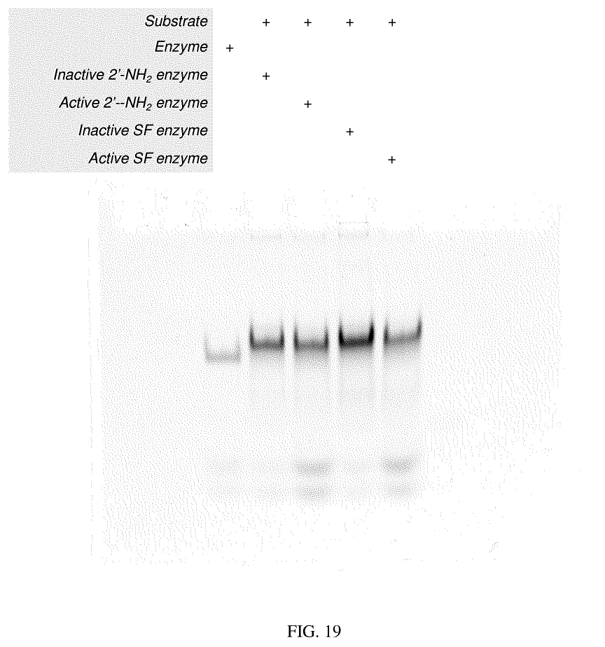

To evaluate the system in an intracellular environment, the DAP-ID platform containing the Na+-DNAzyme was delivered into HepG2 cells. After decaging, both the NCactive,NH2 and the 2′-SF-modified active DNAzyme displayed ˜2-fold fluorescein increases over the inactive sensor controls, indicating the sensor's response to Na+ (FIG. 15 panel B). For protein labeling, only the 2′-SF active DNAzyme displayed a ˜3-fold higher streptavidin signal compared to the NCinactive,SF (FIG. 15 panel B). In contrast, the 2′-NH2 inactive and active DNAzymes did not display significant protein labeling (FIG. 15 panel B). Without light activation, all groups' fluorescence signals remained low (FIG. 18). To ensure labeling was not due to an artifact of incomplete sensor hybridization, it was confirmed that the sensor was completely hybridized through a native gel (FIG. 19).

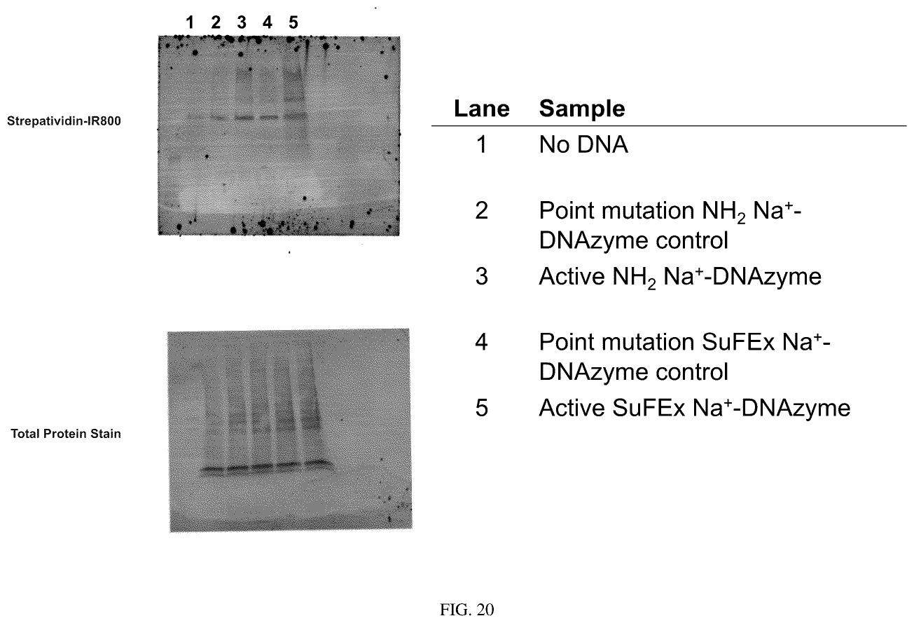

To determine the efficiency of the protein labeling, the above delivery and photoactivation process was repeated into intact cells and the cell lysate analyzed through a Western blot. An increase in biotinylated proteins for the 2′-SF active DNAzyme group was observed, indicating successful Na+-guided protein biotinylation (FIG. 15 panel C). In contrast, the controls did not show significant protein labeling above background endogenous biotinylated carboxylases, although equal amounts of total protein were loaded per lane (FIG. 20) (Rhee et al., 2013). To further evaluate if DAP-ID can label Na+-related proteins, quantitative proteomics was performed. The NCinactive,SF served as the negative control to account for any background labeling due to sensor degradation, dehybridization, or nonspecific interactions with proteins in the absence of Na+. Like the ATP aptamer, three biological replicates were performed with each group, using 1 mg of total protein input, streptavidin bead purification, and on-bead sample preparation. Using TMT-based quantitative mass spectrometry, 87 significantly enriched proteins were identified with the 2′-SF active DNAzyme (FIG. 15 panel D, Table 2).

To evaluate DAP-ID's specific labeling of metabolite-and metal ion-microenvironments, the datasets between the ATP aptamer-guided proteomics was compared with the Na+-DNAzyme-guided proteomics, both of which were performed in the same cell type (HepG2). No overlaps of identified proteins were present, highlighting the platform's tight spatial resolution and protein labeling selectivity, and the existence of metabolite/metal ion microenvironments within the cell. Significantly, 70% of the proteins from the Na+-dependent proximity labeling have been previously validated to interact with Na+ either directly or indirectly through protein-protein interactions with Na+-dependent complexes (FIG. 15 panel D). In the 70% of identified known Na+-protein relationships, these interactions varied greatly in mechanism. Structurally, multiple ribosomal subunits were identified due to high Na+ coordination to mRNA in the ribosome (Klein et al., 2004). In the apolipoprotein family, apolipoprotein A-I were identified, in which Na+ promotes supporting acyltransferase reaction rates through modulation of enzyme-substrate interactions (Jonas et al., 1987), apolipoprotein A-II, in which Na+ results in destabilization of high-density lipoprotein particles (Jayaraman et al., 2006), and apolipoprotein E, in which Na+ modulates ApoE's distal organization (Stuchell-Brereton et al., 2023). Allosterically, complement factors and prothrombin were identified, both of which contain Na+-activated binding pockets (Pineda et al., 2004). Multiple early and recycling endosome protein markers were identified, such as the early Rab family, which corroborates with high early endosomal Na+levels (Zou et al., 2023). As an example of a Na+-interactor, gelsolin was identified-an actin-binding protein known to activate Na+ channels (Cantiello et al., 1991). For cellular compartment analysis, a high degree of nuclear and ribosomal proteins were identified, similar to characterization of metal-binding protein discovery in cell lysates, indicating these cellular regions may be generally metal rich (Zeng et al., 2024).

### Example 4—Validation of a Previously Unknown Na⁺-Protein Interaction

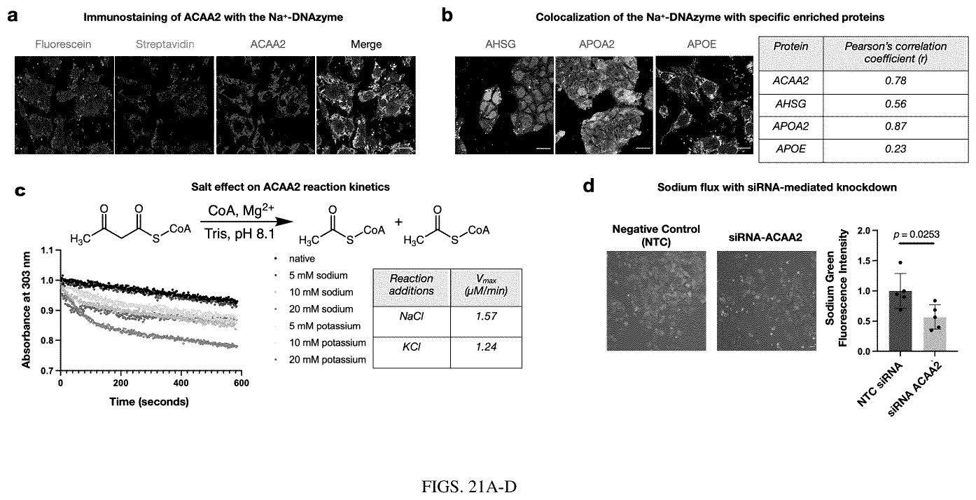

To demonstrate DAP-ID's utility for identifying new proteins associated with metal ions, the effects of Na+ on one of the most significantly enriched Na+-DNAzyme guided protein hits—acetyl coenzyme A acyltransferase 2 (ACAA2) was investigated. ACAA2 is a thiolase that performs fatty acid B-oxidation and synthesizes acetoacetyl-CoA from acetyl-CoA, as a cornerstone in cellular metabolism (Kiema et al., 2014). While limited information on ACAA2's salt relationship is available, other enzymes in this family undergo allosteric modulation by K+ (Haapalainen et al., 2007). To evaluate whether ACAA2 is associated with Na+, 2′-SF Na+-DNAzyme was first delivered into HepG2 cells and then immunofluorescence of ACAA2performed. A high Pearson's correlation coefficient (0.78) of the probe's total labeled proteins with ACAA2 was found (FIG. 21 panel A). Likewise, the correlations with other enriched proteins found positive overlaps between the sensor and protein (0.56 for AHSG, 0.87 for APOA2, and 0.23 for APOE) (FIG. 21 panel B). This wide range of colocalization values between the Na+-DNAzyme and immunostained proteins indicates that DAP-ID does not simply identify the most abundant protein near the delivered sensor.

To determine a functional effect of the sodium pool's proximity with ACAA2, the effect of Na+ on ACAA2's thiolase reaction was investigated. Both Na+ and K+ increased ACAA2's Vmax compared to no salt added, with sodium resulting in the largest Vmax (FIG. 21 panel C). It was next evaluated if the relationship between Na+ and ACAA2 could be studied in live cells as well. After siRNA-mediated knockdown of ACAA2, a significant decrease in intracellular Na+ levels compared to the negative control siRNA using commercially available Sodium Green as an indicator of intracellular sodium levels was detected (FIG. 21 panel D). This result suggests a potential regulatory network between ACAA2, other proteins, and Na+ homeostasis. Overall, these validation experiments verify the system's identification of a previously unknown Na+-related protein that not only coexists in the same microenvironment as Na+, but is also affected by Na+ levels, in which high Na+ concentrations increase enzyme activity.

The platform may exhibit high success rates (i.e., 70-97%) at identifying known interactors. It may may exhibit an ability to distinguish between the unique microenvironments of ATP and Na+, which demonstrates its accuracy in identifying new and previously unknown protein-metabolite/metal ion interactions. To verify DAP-ID's accuracy in identifying new cellular relationships, sodium's relationship to ACAA2 was validated, a significantly enriched hit from the Na+-DNAzyme dataset. Na+ increases ACAA2 thiolase activity in vitro, with regulation of the intracellular Na+ pool after ACAA2 knockdown. The positive validation of ACAA2 exemplifies DAP-ID's potential to correctly identify new metabolite/metal ion-protein relationships.

A more reactive electrophile can be applied for faster kinetics and improved labeling efficiency. Organelle targeting groups can be used to access subcellular organelles, like the inner mitochondria, the Golgi apparatus, and the endoplasmic reticulum. Aptamers or DNAzymes with varying binding affinities for the same target can be used to find metabolite/metal ion-protein interactions of varying strengths.

DAP-ID can serve as a foundation for the initial expansion of the proximal labeling methods for investigating the metabolite/metal ion-protein interactome. It provides unique information as it is performed in live subcellular environments, does not require protein targeting motifs that may bias results toward limited known consensus motifs, and responds to native levels of metabolites and metal ions without the need to dose cells.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Jelcic, M.; Wang, K.; Hui, K. L.; Cai, X.-C.; Enyedi, B.; Luo, M.; Niethammer, P. A Photo-Clickable ATP-Mimetic Reveals Nucleotide Interactors in the Membrane Proteome. Cell Chem. Biol. 2020, 27 (8), 1073-1083.e12. https://doi.org/10.1016/j.chembiol.2020.05.010.
Trefely, S.; Huber, K.; Liu, J.; Noji, M.; Stransky, S.; Singh, J.; Doan, M. T.; Lovell, C. D.; Von Krusenstiern, E.; Jiang, H.; Bostwick, A.; Pepper, H. L.; Izzo, L.; Zhao, S.; Xu, J. P.; Bedi, K. C.; Rame, J. E.; Bogner-Strauss, J. G.; Mesaros, C.; Sidoli, S.; Wellen, K. E.; Snyder, N. W. Quantitative Subcellular Acyl-CoA Analysis Reveals Distinct Nuclear Metabolism and Isoleucine-Dependent Histone Propionylation. Mol. Cell 2022, 82 (2), 447-462.e6. https://doi.org/10.1016/j.molcel.2021.11.006.
Agasid, M. T.; Sørensen, L.; Urner, L. H.; Yan, J.; Robinson, C. V. The Effects of Sodium Ions on Ligand Binding and Conformational States of G Protein-Coupled Receptors—Insights from Mass Spectrometry. J. Am. Chem. Soc. 2021, 143 (11), 4085-4089. https://doi.org/10.1021/jacs.0c11837.
Chin, D.; Means, A. R. Calmodulin: A Prototypical Calcium Sensor. Trends Cell Biol. 2000, 10 (8), 322-328. https://doi.org/10.1016/S0962-8924(00)01800-6.
Dean, K. M.; Qin, Y.; Palmer, A. E. Visualizing Metal Ions in Cells: An Overview of Analytical Techniques, Approaches, and Probes. Biochim. Biophys. Acta BBA—Mol. Cell Res. 2012, 1823 (9), 1406-1415. https://doi.org/10.1016/j.bbamcr.2012.04.001.
Imamura, H.; Huynh Nhat, K. P.; Togawa, H.; Saito, K.; Iino, R.; Kato-Yamada, Y.; Nagai, T.; Noji, H. Visualization of ATP Levels inside Single Living Cells with Fluorescence Resonance Energy Transfer-Based Genetically Encoded Indicators. Proc. Natl. Acad. Sci. 2009, 106 (37), 15651-15656. https://doi.org/10.1073/pnas.0904764106.
Meyer, J.; Untiet, V.; Fahlke, C.; Gensch, T.; Rose, C. R. Quantitative Determination of Cellular [Na+] by Fluorescence Lifetime Imaging with CoroNaGreen. J. Gen. Physiol. 2019, 151 (11), 1319-1331. https://doi.org/10.1085/jgp.201912404.
Cho, K. F.; Branon, T. C.; Udeshi, N. D.; Myers, S. A.; Carr, S. A.; Ting, A. Y. Proximity Labeling in Mammalian Cells with TurboID and Split-TurboID. Nat. Protoc. 2020, 15 (12), 3971-3999. https://doi.org/10.1038/s41596-020-0399-0.
Rhee, H.-W.; Zou, P.; Udeshi, N. D.; Martell, J. D.; Mootha, V. K.; Carr, S. A.; Ting, A. Y. Proteomic Mapping of Mitochondria in Living Cells via Spatially-Restricted Enzymatic Tagging. Science 2013, 339 (6125), 1328-1331. https://doi.org/10.1126/science.1230593.
Geri, J. B.; Oakley, J. V.; Reyes-Robles, T.; Wang, T.; McCarver, S. J.; White, C. H.; Rodriguez-Rivera, F. P.; Parker, D. L.; Hett, E. C.; Fadeyi, O. O.; Oslund, R. C.; MacMillan, D. W. C. Microenvironment Mapping via Dexter Energy Transfer on Immune Cells. Science 2020, 367 (6482), 1091-1097. https://doi.org/10.1126/science.aay4106.
Roux, K. J.; Kim, D. I.; Raida, M.; Burke, B. A Promiscuous Biotin Ligase Fusion Protein Identifies Proximal and Interacting Proteins in Mammalian Cells. J. Cell Biol. 2012, 196 (6), 801-810. https://doi.org/10.1083/jcb.201112098.
Padrón, A.; Iwasaki, S.; Ingolia, N. T. Proximity RNA Labeling by APEX-Seq Reveals the Organization of Translation Initiation Complexes and Repressive RNA Granules. Mol. Cell 2019, 75 (4), 875-887.e5. https://doi.org/10.1016/j.molcel.2019.07.030.
Wei, X.; Li, S.; Hu, J. A TurboID-Based Proximity Labelling Approach for Identifying the DNA-Binding Proteins. STAR Protoc. 2023, 4 (1), 102139. https://doi.org/10.1016/j.xpro.2023.102139.
Kim, J. W.; Yong, A. J. H.; Aisenberg, E. E.; Lobel, J. H.; Wang, W.; Dawson, T. M.; Dawson, V. L.; Gao, R.; Jan, Y. N.; Bateup, H. S.; Ingolia, N. T. Molecular Recording of Calcium Signals via Calcium-Dependent Proximity Labeling. Nat. Chem. Biol. 2024. https://doi.org/10.1038/s41589-024-01603-7.
Hicks, K. G.; Cluntun, A. A.; Schubert, H. L.; Hackett, S. R.; Berg, J. A.; Leonard, P. G.; Ajalla Aleixo, M. A.; Zhou, Y.; Bott, A. J.; Salvatore, S. R.; Chang, F.; Blevins, A.; Barta, P.; Tilley, S.; Leifer, A.; Guzman, A.; Arok, A.; Fogarty, S.; Winter, J. M.; Ahn, H.-C.; Allen, K. N.; Block, S.; Cardoso, I. A.; Ding, J.; Dreveny, I.; Gasper, W. C.; Ho, Q.; Matsuura, A.; Palladino, M. J.; Prajapati, S.; Sun, P.; Tittmann, K.; Tolan, D. R.; Unterlass, J.; VanDemark, A. P.; Vander Heiden, M. G.; Webb, B. A.; Yun, C.-H.; Zhao, P.; Wang, B.; Schopfer, F. J.; Hill, C. P.; Nonato, M. C.; Muller, F. L.; Cox, J. E.; Rutter, J. Protein-Metabolite Interactomics of Carbohydrate Metabolism Reveal Regulation of Lactate Dehydrogenase. Science 2023, 379 (6636), 996-1003. https://doi.org/10.1126/science.abm3452.
Piazza, I.; Kochanowski, K.; Cappelletti, V.; Fuhrer, T.; Noor, E.; Sauer, U.; Picotti, P. A Map of Protein-Metabolite Interactions Reveals Principles of Chemical Communication. Cell 2018, 172 (1-2), 358-372.e23. https://doi.org/10.1016/j.cell.2017.12.006.
Zeng, X.; Wei, T.; Wang, X.; Liu, Y.; Tan, Z.; Zhang, Y.; Feng, T.; Cheng, Y.; Wang, F.; Ma, B.; Qin, W.; Gao, C.; Xiao, J.; Wang, C. Discovery of Metal-Binding Proteins by Thermal Proteome Profiling. Nat. Chem. Biol. 2024. https://doi.org/10.1038/s41589-024-01563-y.
Qin, W.; Yang, F.; Wang, C. Chemoproteomic Profiling of Protein-Metabolite Interactions. Curr. Opin. Chem. Biol. 2020, 54, 28-36. https://doi.org/10.1016/j.cbpa.2019.11.003.
Lindsley, J. E.; Rutter, J. Whence Cometh the Allosterome? Proc. Natl. Acad. Sci. 2006, 103 (28), 10533-10535. https://doi.org/10.1073/pnas.0604452103.
Adachi, J.; Kishida, M.; Watanabe, S.; Hashimoto, Y.; Fukamizu, K.; Tomonaga, T. Proteome-Wide Discovery of Unknown ATP-Binding Proteins and Kinase Inhibitor Target Proteins Using an ATP Probe. J. Proteome Res. 2014, 13 (12), 5461-5470. https://doi.org/10.1021/pr500845u.
Milione, R. R.; Schell, B.-B.; Douglas, C. J.; Seath, C. P. Creative Approaches Using Proximity Labeling to Gain New Biological Insights. Trends Biochem. Sci. 2024, 49 (3), 224-235. https://doi.org/10.1016/j.tibs.2023.12.005.
Hanswillemenke, A.; Hofacker, D. T.; Sorgenfrei, M.; Fruhner, C.; Franz-Wachtel, M.; Schwarzer, D.; Maček, B.; Stafforst, T. Profiling the Interactome of Oligonucleotide Drugs by Proximity Biotinylation. Nat. Chem. Biol. 2024, 20 (5), 555-565. https://doi.org/10.1038/s41589-023-01530-z.
Pani, S.; Qiu, T.; Kentala, K.; Azizi, S.-A.; Dickinson, B. C. Bioorthogonal Masked Acylating Agents for Proximity-Dependent RNA Labelling. Nat. Chem. 2024, 16 (5), 717-726. https://doi.org/10.1038/s41557-024-01493-1.
Myers, S. A.; Wright, J.; Peckner, R.; Kalish, B. T.; Zhang, F.; Carr, S. A. Discovery of Proteins Associated with a Predefined Genomic Locus via dCas9-APEX-Mediated Proximity Labeling. Nat. Methods 2018, 15 (6), 437-439. https://doi.org/10.1038/s41592-018-0007-1.

Huizenga, D. E.; Szostak, J. W. A DNA Aptamer That Binds Adenosine and ATP. Biochemistry 1995, 34 (2), 656-665. https://doi.org/10.1021/bi00002a033.
Yu, H.; Alkhamis, O.; Canoura, J.; Liu, Y.; Xiao, Y. Advances and Challenges in Small-Molecule DNA Aptamer Isolation, Characterization, and Sensor Development. Angew. Chem. Int. Ed. 2021, 60 (31), 16800-16823. https://doi.org/10.1002/anie.202008663.
Nakatsuka, N.; Yang, K.-A.; Abendroth, J. M.; Cheung, K. M.; Xu, X.; Yang, H.; Zhao, C.; Zhu, B.; Rim, Y. S.; Yang, Y.; Weiss, P. S.; Stojanović, M. N.; Andrews, A. M. Aptamer-Field-Effect Transistors Overcome Debye Length Limitations for Small-Molecule Sensing. Science 2018, 362 (6412), 319-324. https://doi.org/10.1126/science.aao6750.
Xie, S.; Du, Y.; Zhang, Y.; Wang, Z.; Zhang, D.; He, L.; Qiu, L.; Jiang, J.; Tan, W. Aptamer-Based Optical Manipulation of Protein Subcellular Localization in Cells. Nat. Commun. 2020, 11 (1), 1347. https://doi.org/10.1038/s41467-020-15113-2.
Breaker, R. R.; Joyce, G. F. A DNA Enzyme That Cleaves RNA. Chem. Biol. 1994, 1 (4), 223-229. https://doi.org/10.1016/1074-5521 (94) 90014-0.
McGhee, C. E.; Yang, Z.; Guo, W.; Wu, Y.; Lyu, M.; DeLong, C. J.; Hong, S.; Ma, Y.; McInnis, M. G.; O'Shea, K. S.; Lu, Y. DNAzyme-Based Lithium-Selective Imaging Reveals Higher Lithium Accumulation in Bipolar Disorder Patient-Derived Neurons. ACS Cent. Sci. 2021, 7 (11), 1809-1820. https://doi.org/10.1021/acscentsci.1c00843.
Torabi, S.-F.; Wu, P.; McGhee, C. E.; Chen, L.; Hwang, K.; Zheng, N.; Cheng, J.; Lu, Y. In Vitro Selection of a Sodium-Specific DNAzyme and Its Application in Intracellular Sensing. Proc. Natl. Acad. Sci. 2015, 112 (19), 5903-5908. https://doi.org/10.1073/pnas.1420361112.
Li, J.; Zhang, Z.; Liu, R.; Amini, R.; Salena, B. J.; Li, Y. Discovery and Translation of Functional Nucleic Acids for Clinically Diagnosing Infectious Diseases: Opportunities and Challenges. TrAC Trends Anal. Chem. 2023, 158, 116886. https://doi.org/10.1016/j.trac.2022.116886.
Zhou, W.; Saran, R.; Liu, J. Metal Sensing by DNA. Chem. Rev. 2017, 117 (12), 8272-8325. https://doi.org/10.1021/acs.chemrev.7b00063.
Munzar, J. D.; Ng, A.; Juncker, D. Comprehensive Profiling of the Ligand Binding Landscapes of Duplexed Aptamer Families Reveals Widespread Induced Fit. Nat. Commun. 2018, 9 (1), 343. https://doi.org/10.1038/s41467-017-02556-3.
Barrow, A. S.; Smedley, C. J.; Zheng, Q.; Li, S.; Dong, J.; Moses, J. E. The Growing Applications of SuFEx Click Chemistry. Chem. Soc. Rev. 2019, 48 (17), 4731-4758. https://doi.org/10.1039/C8CS00960K.
Qin, Z.; Zhang, K.; He, P.; Zhang, X.; Xie, M.; Fu, Y.; Gu, C.; Zhu, Y.; Tong, A.; Wei, H.; Zhang, C.; Xiang, Y. Discovering Covalent Inhibitors of Protein-Protein Interactions from Trillions of
Sulfur (VI) Fluoride Exchange-Modified Oligonucleotides. Nat. Chem. 2023, 15 (12), 1705-1714. https://doi.org/10.1038/s41557-023-01304-z.
Gould, N. S.; Evans, P.; Martínez-Acedo, P.; Marino, S. M.; Gladyshev, V. N.; Carroll, K. S.; Ischiropoulos, H. Site-Specific Proteomic Mapping Identifies Selectively Modified Regulatory Cysteine Residues in Functionally Distinct Protein Networks. Chem. Biol. 2015, 22 (7), 965-975. https://doi.org/10.1016/j.chembiol.2015.06.010.
Hong, S.; Zhang, X.; Lake, R. J.; Pawel, G. T.; Guo, Z.; Pei, R.; Lu, Y. A Photo-Regulated Aptamer Sensor for Spatiotemporally Controlled Monitoring of ATP in the Mitochondria of Living Cells. Chem. Sci. 2020, 11 (3), 713-720. https://doi.org/10.1039/C9SC04773E.
Zhang, Q.-L.; Wang, L.-L.; Liu, Y.; Lin, J.; Xu, L. A Kinetically Controlled Platform for Ligand-Oligonucleotide Transduction. Nat. Commun. 2021, 12 (1), 4654. https://doi.org/10.1038/s41467-021-24962-4.
Shi, Y.; Yun, Y.; Wang, R.; Liu, Z.; Wu, Z.; Xiang, Y.; Zhang, J. Engineering Covalent Aptamer Chimeras for Enhanced Autophagic Degradation of Membrane Proteins. Angew. Chem. Int. Ed. 2025, e202425123. https://doi.org/10.1002/anie.202425123.
Benjamini, Y.; Hochberg, Y. Controlling the False Discovery Rate: A Practical and Powerful Approach to Multiple Testing. J. R. Stat. Soc. Ser. B Stat. Methodol. 1995, 57 (1), 289-300. https://doi.org/10.1111/j.2517-6161.1995.tb02031.x.
Kim, S. H.; Lin, R. J. Pre-mRNA Splicing within an Assembled Yeast Spliceosome Requires an RNA-Dependent ATPase and ATP Hydrolysis. Proc. Natl. Acad. Sci. 1993, 90 (3), 888-892. https://doi.org/10.1073/pnas.90.3.888.
Onishi, H.; Kino, Y.; Morita, T.; Futai, E.; Sasagawa, N.; Ishiura, S. MBNL1 Associates with YB-1 in Cytoplasmic Stress Granules. J. Neurosci. Res. 2008, 86 (9), 1994-2002. https://doi.org/10.1002/jnr.21655.
Miranda, G. A.; Chokler, I.; Aguilera, R. J. The Murine Nucleolin Protein Is an Inducible DNA and ATP Binding Protein Which Is Readily Detected in Nuclear Extracts of Lipopolysaccharide-Treated Splenocytes. Exp. Cell Res. 1995, 217 (2), 294-308. https://doi.org/10.1006/excr.1995.1090.
Puri, T.; Wendler, P.; Sigala, B.; Saibil, H.; Tsaneva, I. R. Dodecameric Structure and ATPase Activity of the Human TIP48/TIP49 Complex. J. Mol. Biol. 2007, 366 (1), 179-192. https://doi.org/10.1016/j.jmb.2006.11.030.
Miki, H.; Setou, M.; Kaneshiro, K.; Hirokawa, N. All Kinesin Superfamily Protein, KIF, Genes in Mouse and Human. Proc. Natl. Acad. Sci. 2001, 98 (13), 7004-7011. https://doi.org/10.1073/pnas.111145398.
Hanks, S. K.; Quinn, A. M.; Hunter, T. The Protein Kinase Family: Conserved Features and Deduced Phylogeny of the Catalytic Domains. Science 1988, 241 (4861), 42-52. https://doi.org/10.1126/science.3291115.
Rouaud, F.; Huang, W.; Flinois, A.; Jain, K.; Vasileva, E.; Di Mattia, T.; Mauperin, M.; Parry, D. A. D.; Dugina, V.; Chaponnier, C.; Méan, I.; Montessuit, S.; Mutero-Maeda, A.; Yan, J.; Citi, S. Cingulin and Paracingulin Tether Myosins-2 to Junctions to Mechanoregulate the Plasma Membrane. J. Cell Biol. 2023, 222 (7), e202208065. https://doi.org/10.1083/jcb.202208065.
Hein, M. Y.; Hubner, N. C.; Poser, I.; Cox, J.; Nagaraj, N.; Toyoda, Y.; Gak, I. A.; Weisswange, I.; Mansfeld, J.; Buchholz, F.; Hyman, A. A.; Mann, M. A Human Interactome in Three Quantitative Dimensions Organized by Stoichiometries and Abundances. Cell 2015, 163 (3), 712-723. https://doi.org/10.1016/j.cell.2015.09.053.
Nayler, O. SAF-B Protein Couples Transcription and Pre-mRNA Splicing to SAR/MAR Elements. Nucleic Acids Res. 1998, 26 (15), 3542-3549. https://doi.org/10.1093/nar/26.15.3542.
Martin, I. M.; Nava, M. M.; Wickström, S. A.; Gräter, F. ATP Allosterically Stabilizes Integrin-Linked Kinase for Efficient Force Generation. Proc. Natl. Acad. Sci. 2022, 119 (11), e2106098119. https://doi.org/10.1073/pnas.2106098119.
Wiśniewski, J. R.; Vildhede, A.; Norén, A.; Artursson, P. In-Depth Quantitative Analysis and Comparison of the Human Hepatocyte and Hepatoma Cell Line HepG2 Proteomes. J. Proteomics 2016, 136, 234-247. https://doi.org/10.1016/j.jprot.2016.01.016.
Lukacs, G. L.; Haggie, P.; Seksek, O.; Lechardeur, D.; Freedman, N.; Verkman, A. S. Size-Dependent DNA Mobility in Cytoplasm and Nucleus. J. Biol. Chem. 2000, 275 (3), 1625-1629. https://doi.org/10.1074/jbc.275.3.1625.
Leathers, A.; Walczak, M.; Brady, R. A.; Al Samad, A.; Kotar, J.; Booth, M. J.; Cicuta, P.; Di Michele, L. Reaction-Diffusion Patterning of DNA-Based Artificial Cells. J. Am. Chem. Soc. 2022, 144 (38), 17468-17476. https://doi.org/10.1021/jacs.2c06140.
Yang, B.; Wu, H.; Schnier, P. D.; Liu, Y.; Liu, J.; Wang, N.; DeGrado, W. F.; Wang, L. Proximity-Enhanced SuFEx Chemical Cross-Linker for Specific and Multitargeting Cross-Linking Mass Spectrometry. Proc. Natl. Acad. Sci. 2018, 115 (44), 11162-11167. https://doi.org/10.1073/pnas.1813574115.
Luy, J.-N.; Tonner, R. Complementary Base Lowers the Barrier in SuFEx Click Chemistry for Primary Amine Nucleophiles. ACS Omega 2020, 5 (48), 31432-31439. https://doi.org/10.1021/acsomega.0c05049.
Klein, D. J.; Moore, P. B.; Steitz, T. A. The Contribution of Metal Ions to the Structural Stability of the Large Ribosomal Subunit. RNA 2004, 10 (9), 1366-1379. https://doi.org/10.1261/rna.7390804.
Jonas, A.; Zorich, N. L.; Kézdy, K. E.; Trick, W. E. Reaction of Discoidal Complexes of Apolipoprotein A-I and Various Phosphatidylcholines with Lecithin Cholesterol Acyltransferase.
Interfacial Effects. J. Biol. Chem. 1987, 262 (9), 3969-3974. https://doi.org/10.1016/S0021-9258(18)61297-9.
Jayaraman, S.; Gantz, D. L.; Gursky, O. Effects of Salt on the Thermal Stability of Human Plasma High-Density Lipoprotein. Biochemistry 2006, 45 (14), 4620-4628. https://doi.org/10.1021/bi0524565.
Stuchell-Brereton, M. D.; Zimmerman, M. I.; Miller, J. J.; Mallimadugula, U. L.; Incicco, J. J.; Roy, D.; Smith, L. G.; Cubuk, J.; Baban, B.; DeKoster, G. T.; Frieden, C.; Bowman, G. R.; Soranno, A. Apolipoprotein E4 Has Extensive Conformational Heterogeneity in Lipid-Free and Lipid-Bound Forms. Proc. Natl. Acad. Sci. 2023, 120 (7), e2215371120. https://doi.org/10.1073/pnas.2215371120.
Pineda, A. O.; Carrell, C. J.; Bush, L. A.; Prasad, S.; Caccia, S.; Chen, Z.-W.; Mathews, F. S.; Di Cera, E. Molecular Dissection of Na+Binding to Thrombin. J. Biol. Chem. 2004, 279 (30), 31842-31853. https://doi.org/10.1074/jbc.M401756200.
Zou, J.; Mitra, K.; Anees, P.; Oettinger, D.; Ramirez, J. R.; Veetil, A. T.; Gupta, P. D.; Rao, R.; Smith, J. J.; Kratsios, P.; Krishnan, Y. A DNA Nanodevice for Mapping Sodium at Single-Organelle Resolution. Nat. Biotechnol. 2023. https://doi.org/10.1038/s41587-023-01950-1.
Cantiello, H. F.; Stow, J. L.; Prat, A. G.; Ausiello, D. A. Actin Filaments Regulate Epithelial Na+ Channel Activity. Am. J. Physiol.-Cell Physiol. 1991, 261 (5), C882-C888. https://doi.org/10.1152/ajpcell.1991.261.5.C882.
Kiema, T.-R.; Harijan, R. K.; Strozyk, M.; Fukao, T.; Alexson, S. E. H.; Wierenga, R. K. The Crystal Structure of Human Mitochondrial 3-Ketoacyl-CoA Thiolase (T1): Insight into the Reaction Mechanism of Its Thiolase and Thioesterase Activities. Acta Crystallogr. D Biol. Crystallogr. 2014, 70 (12), 3212-3225. https://doi.org/10.1107/S1399004714023827.
Haapalainen, A. M.; Meriläinen, G.; Pirilä, P. L.; Kondo, N.; Fukao, T.; Wierenga, R. K. Crystallographic and Kinetic Studies of Human Mitochondrial Acetoacetyl-CoA Thiolase: The Importance of Potassium and Chloride Ions for Its Structure and Function. Biochemistry 2007, 46 (14), 4305-4321. https://doi.org/10.1021/bi6026192.
Chaulk, S. G.; MacMillan, A. M. Synthesis of Oligo-RNAs with Photocaged Adenosine 2′-Hydroxyls. Nat Protoc 2007, 2 (5), 1052-1058. https://doi.org/10.1038/nprot.2007.154.
Kong, A. T.; Leprevost, F. V.; Avtonomov, D. M.; Mellacheruvu, D.; Nesvizhskii, A. I. MSFragger: Ultrafast and Comprehensive Peptide Identification in Mass Spectrometry-Based Proteomics. Nat Methods 2017, 14 (5), 513-520. https://doi.org/10.1038/nmeth.4256.
Kang, M.; Day, C. A.; Kenworthy, A. K.; DiBenedetto, E. Simplified Equation to Extract Diffusion Coefficients from Confocal FRAP Data. Traffic 2012, 13 (12), 1589-1600. https://doi.org/10.1111/tra.12008.

1. A method of detecting macromolecule interactors in the vicinity of a metabolite or metal ion pool in a cell, the method comprising:
(a) administering to the cell a conformationally gated sensor that has affinity for the metabolite or metal ion, wherein the conformationally gated sensor comprises a hidden reactive electrophile;
(b) incubating the cell under conditions to allow the sensor to interact with the metabolite or metal ion, thereby undergoing a conformational change upon binding the metabolite or metal ion, thereby exposing the reactive electrophile, wherein the exposed reactive electrophile can label nearby macromolecules; and
(c) extracting and identifying the proximal macromolecules that have been labeled by the sensor's reactive electrophile.

2. The method of claim 1, wherein the conformationally gated sensor is an aptamer.

3. The method of claim 2, wherein the aptamer comprises a fluorescent tag.

4. The method of claim 3, wherein the fluorescent tag is fluorescein.

5. The method of claim 2, further comprising a photocaged strand complementary to the aptamer, and wherein the photocaged complementary strand comprises a photocleavable o-nitrobenzyl group, and wherein the aptamer hybridizes with the photocaged strand generating a protected aptamer to prevent the aptamer from premature response to the metabolite or metal ions, and to protect the reactive electrophile from covalent labeling of reactive nucleophiles.

6. The method of claim 5, wherein the photocaged complementary strand comprises a quencher.

7. The method of claim 5, wherein the method further comprises exposing the protected aptamer to a light stimulus, wherein the exposing to the light stimulus decages the aptamer, thereby allowing binding of the aptamer to the metabolite or metal ion.

8. The method of claim 1, wherein the conformationally gated sensor is a DNAzyme, wherein the DNAzyme comprises an enzyme strand and a substrate strand.

9. The method of claim 8, wherein the substrate strand comprises a photocleavable o-nitrobenzyl group.

10. The method of claim 9, wherein the method further comprises exposing the DNAzyme to a light stimulus, wherein the exposing to the light stimulus decages the substrate strand, thereby allowing cleavage of the substrate strand upon binding of the metabolite or metal ion.

11. The method of claim 8, wherein the substrate strand comprises at least one ribonucleotide.

12. The method of claim 8, wherein binding of the metabolite or metal ion leads to cleavage of the substrate strand by the enzyme strand, whereby the cleavage leads to exposure of the reactive electrophile.

13. The method of claim 8, wherein the enzyme strand comprises a fluorescent tag.

14. The method of claim 13, wherein the fluorescent tag is fluorescein.

15. The method of claim 8, wherein the substrate strand comprises a quencher.

16. The method of claim 1, wherein the hidden reactive electrophile is an electrophilic sulfonyl fluoride group.

17. The method of claim 16, wherein the electrophilic sulfonyl fluoride group is at the 2′ sugar position of a DNA base in the sensor.

18. The method of claim 1, wherein the conformationally gated sensor comprises a biotin.

19. The method of claim 18, wherein extracting the labeled macromolecules comprises pulling down the labeled proteins using streptavidin.

20. The method of claim 1, wherein identifying the proximal macromolecules comprises quantitative mass spectrometry.


SEQUENCE LISTING (18 sequences)

SEQ ID NO: 1 - DNA, 58 bp, synthetic construct
  source 1..58: mol_type = other DNA
  modified_base 6: mod_base = OTHER; note = biotin thymine
  modified_base 56..57: mod_base = OTHER; note = 2'-NH2 cytosine
ggcggtaccaggtcaaaggtgggtgaggggacgccaagagtccccgcggttagatcca

SEQ ID NO: 2 - DNA, 58 bp, synthetic construct
  source 1..58: mol_type = other DNA
  modified_base 6: mod_base = OTHER; note = biotin thymine
  modified_base 56..57: mod_base = OTHER; note = 2'-NH2 cytosine
ggcggtaccaggacaaaggtgggtgaggggacgccaagagtccccgcggttagatcca

SEQ ID NO: 3 - DNA, 58 bp, synthetic construct
  source 1..58: mol_type = other DNA
  modified_base 6: mod_base = OTHER; note = biotin thymine
  modified_base 56..57: mod_base = OTHER; note = 2'-aryl sulfonyl fluoride cytosine
ggcggtaccaggtcaaaggtgggtgaggggacgccaagagtccccgcggttacatcca

SEQ ID NO: 4 - DNA, 58 bp, synthetic construct
  source 1..58: mol_type = other DNA
  modified_base 6: mod_base = OTHER; note = biotin thymine
  modified_base 56..57: mod_base = OTHER; note = 2'-aryl sulfonyl fluoride cytosine
ggcggtaccaggacaaaggtgggtgaggggacgccaagagtccccgcggttacatcca

SEQ ID NO: 5 - DNA, 22 bp, synthetic construct
  source 1..22: mol_type = other DNA
  modified_base 10: mod_base = OTHER; note = 2'-O-Nitrobenzyl adenine
tggatgtataggaagtaccgcc

SEQ ID NO: 6 - DNA, 32 bp, synthetic construct
  source 1..32: mol_type = other DNA
  modified_base 1: mod_base = OTHER; note = fluorescein thymine
  modified_base 3: mod_base = OTHER; note = 2'-NH2 uracil
  modified_base 5: mod_base = OTHER; note = 2'-NH2 uracil
tgtgtacctgggggagtattgcggaggaaggt

SEQ ID NO: 7 - DNA, 32 bp, synthetic construct; identical to SEQ ID NO: 6
  source 1..32: mol_type = other DNA
  modified_base 1: mod_base = OTHER; note = fluorescein thymine
  modified_base 3: mod_base = OTHER; note = 2'-aryl sulfonyl fluoride uracil
  modified_base 5: mod_base = OTHER; note = 2'-aryl sulfonyl fluoride uracil

SEQ ID NO: 8 - DNA, 32 bp, synthetic construct
  source 1..32: mol_type = other DNA
  modified_base 1: mod_base = OTHER; note = fluorescein thymine
  modified_base 3: mod_base = OTHER; note = 2'-NH2 uracil
  modified_base 5: mod_base = OTHER; note = 2'-NH2 uracil
tgtgtgattgggggaagaacctggagcggttg

SEQ ID NO: 9 - DNA, 32 bp, synthetic construct; identical to SEQ ID NO: 8
  source 1..32: mol_type = other DNA
  modified_base 1: mod_base = OTHER; note = fluorescein thymine
  modified_base 3: mod_base = OTHER; note = 2'-aryl sulfonyl fluoride uracil
  modified_base 5: mod_base = OTHER; note = 2'-aryl sulfonyl fluoride uracil

SEQ ID NO: 10 - DNA, 16 bp, synthetic construct
  source 1..16: mol_type = other DNA
  misc_feature 16: note = the adenosine is attached through the linker [4-(4,4'-dimethoxytrityloxy)butyramidomethyl)-1-(2-nitroph enyl)-ethyl]-2-cyanoethyl-(N,N-diisopropyl)-phosphoramidit e to another nucleic acid sequence
accttcctccgcaata

SEQ ID NO: 11 - DNA, 16 bp, synthetic construct
  source 1..16: mol_type = other DNA
  misc_feature 1: note = the cytosine is attached through the linker [4-(4,4'-dimethoxytrityloxy)butyramidomethyl)-1-(2-nitroph enyl)-ethyl]-2-cyanoethyl-(N,N-diisopropyl)-phosphoramidit e to another nucleic acid
ctcccccaggtacaca

SEQ ID NO: 12 - DNA, 16 bp, synthetic construct
  source 1..16: mol_type = other DNA
  misc_feature 16: note = the cytosine is attached through the linker [4-(4,4'-dimethoxytrityloxy)butyramidomethyl)-1-(2-nitroph enyl)-ethyl]-2-cyanoethyl-(N,N-diisopropyl)-phosphoramidit e to another nucleic acid
caaccgctccaggttc

SEQ ID NO: 13 - DNA, 16 bp, synthetic construct
  source 1..16: mol_type = other DNA
  misc_feature 1: note = the thymine is attached through the linker [4-(4,4' -dimethoxytrityloxy)butyramidomethyl)-1-(2-nitrophenyl)-et hyl]-2-cyanoethyl-(N,N-diisopropyl)-phosphoramidite to another nucleic acid
ttcccccaatcacaca

SEQ ID NO: 14 - RNA, 25 nt, synthetic construct
  source 1..25: mol_type = other RNA
ggatcagatatcaagctggaagatt

SEQ ID NO: 15 - RNA, 27 nt, synthetic construct
  source 1..27: mol_type = other RNA
aatcttccagcttgatatctgatccaa

SEQ ID NO: 16 - DNA, 10 bp, synthetic construct
  source 1..10: mol_type = other DNA
tggatgtata

SEQ ID NO: 17 - DNA, 12 bp, synthetic construct
  source 1..12: mol_type = other DNA
ggaagtaccgcc

SEQ ID NO: 18 - DNA, 58 bp, synthetic construct; identical to SEQ ID NO: 3
  source 1..58: mol_type = other DNA